(19) United States
(12) Patent Application Publication
(10) Pub. No.: US 2013/0116284 A1
(43) Pub. Date: May 9, 2013

(54) LIPOIC ACID AND NITROXIDE DERIVATIVES AND USES THEREOF

(71) Applicant: Andrew Lurie Salzman, West Tisbury, MA (US)

(72) Inventor: Andrew Lurie Salzman, West Tisbury, MA (US)

(73) Assignee: RADIKAL THERAPEUTICS INC., West Tisbury, MA (US)

(21) Appl. No.: 13/697,223

(22) PCT Filed: May 9, 2011
(86) PCT No.: PCT/IL2011/000370

§ 371 Date: Jan. 18, 2013

(30) Foreign Application Priority Data
May 10, 2010 (US) 61333072

(51) Int. Cl. A61K 31/454; A61K 31/4184; C07D 401/14; C07D 409/14; C07D 403/14; C07D 409/06; A61K 31/416

(52) U.S. Cl. CPC A61K 31/454; C07D 409/06; A61K 31/4184; A61K 31/416; C07D 409/14; C07D 403/14; C07D 401/14
USPC 514/322; 548/304.7; 514/394; 548/362.5; 514/406; 546/199

(57) ABSTRACT

Provided are bifunctional compounds comprising a poly(ADP-ribose) polymerase (PARP) inhibitor moiety and a reactive oxygen species (ROS) scavenger moiety, more particularly, a lipoic acid or cyclic nitroxide derivative, covalently attached either directly or via a linker, as well as pharmaceutical compositions comprising them. The compounds and pharmaceutical compositions are useful for prevention, treatment, or management of diseases, disorders and conditions associated with elevated PARP activity or expression.

### TECHNICAL FIELD

The present invention relates to lipoic acid and nitroxide derivatives of poly(ADP-ribose) polymerase (PARP) inhibitors and to pharmaceutical compositions comprising them. The compounds are useful for prevention, treatment, or management of diseases, disorders and conditions associated with elevated PARP activity or expression.

### BACKGROUND ART

Free radicals and other reactive oxygen species (ROS) contribute to the pathogenesis of disease via a number of parallel mechanisms of injury. Recent data indicate that one such mechanism involves the activation of the nuclear DNA-repair enzyme poly(ADP-ribose) polymerase (PARP), a protein whose activation induces cell death and inflammation. It is recognized, for instance, that the oxygen-centered free radical superoxide anion may combine with nitrogen-centered free radical nitric oxide (NO) to form the highly toxic nitrosating species peroxynitrite. In turn, peroxynitrite may induce DNA single strand breakage that activates PARP, which in turn catalyzes the formation of ADP-ribose polymers from NAD⁺, which are covalently attached to nuclear acceptor proteins. PARP activity has been implicated in the regulation of many inflammatory mediators, including effects on the expression of inducible nitric oxide synthase, intercellular adhesion molecule-1 (ICAM-1), and major histocompatibility complex II. PARP activation has also been shown to act as a co-activator in pro-inflammatory transcriptional activation regulated by nuclear factor-kappaB (NF-kB). Severe and prolonged PARP activation may result in substantial depletion of its substrate, NAD⁺, resulting in exhaustion of adenosine triphosphate (ATP), cell death, and necrosis.

Although superoxide anion can be regarded as complicit in the activation of PARP through the generation of peroxynitrite, it has numerous other actions that are independent of PARP activation. Similarly, peroxynitrite has multiple toxic actions, only some of which are dependent upon DNA damage and subsequent PARP activation. Accordingly, blockade of PARP inhibition is only partially effective in experimental models of inflammation, ischemia/reperfusion, or prolonged oxidative stress. Additionally, because superoxide anion is only one of many activators of PARP, its removal partially reduces but may not fully eliminate PARP activation.

Given the parallel nature of many pathologic mechanisms involving free radicals, ROS and PARP activation, a more substantial benefit may accrue from the concomitant inhibition of both PARP activity and redox stress. This effect would be maximized if both these inhibitory actions co-localized in space and time. However, such co-localization would be unlikely to occur by the co-administration of two distinct drugs, such as separate PARP inhibitor and anti-oxidant molecules, because of the unpredictability of their tissue distribution, metabolism, clearance, excretion, and intracellular localization.

### SUMMARY OF INVENTION

According to the present invention, the unpredictability associated with co-administration of a PARP inhibitor and an anti-oxidant molecule as two distinct drugs is overcome by covalently linking these two drugs so that they act as a single bifunctional unit and provide both chemical activities at a similar location and time. Moreover, in view of the fact that this bifunctionality targets two or more points along a step-wise progression of biological reactions, i.e., a biological pathway or “cascade”, it is expected that these bifunctional molecules will act to achieve greater potency and therapeutic ratio than the two drugs separately.

The anti-oxidant moiety to be covalently attached to the PARP inhibitor may be lipoic acid, which is long thought to have anti-oxidant effects and has shown beneficial effects in a variety of diseases, a structural derivative thereof or an analog thereof. Lipoic acid is an organosulfur compound containing two vicinal sulfur atoms (at C6 and C8) attached via a disulfide bond. The carbon atom at C6 is chiral and the molecule exists as two enantiomers, i.e., R-(+)-lipoic acid and S-(−)-lipoic acid, and as a racemic mixture R/S-lipoic acid. Only the R-(+)-enantiomer exists in nature and is an essential co-factor of mitochondrial enzyme complexes. Both free lipoic acid and its reduced form dihydrolipoic acid (DHLA) have been detected within cells after administration of lipoic acid.

Alternatively, the anti-oxidant moiety may be a cyclic nitroxide, which have been variously reported to detoxify a broad spectrum of ROS. Preferred examples of cyclic nitroxides are pyrrolidine- and piperidine-based nitroxides as well as their corresponding reduced hydroxylamine forms, but in particular 2,2,5,5-tetramethylpyrrolidine 1-oxide and 2,2,6,6-tetramethylpiperidine 1-oxide.

A particular such bifunctional compound exemplified here is 2-(4-(1,2-dithiolan-3-yl)butyl)-1H-benzo[d]imidazole-4-carboxamide, which has been found to be a potent PARP inhibitor with IC₅₀ of 26.20 nM and to represent a new class of highly innovative cytoprotective agents, confirmed both in vitro and in vivo to exhibit a remarkable potency.

In one aspect, the present invention thus relates to a compound of the general formula:

A-X—B

or an enantiomer, diastereomer, racemate, or a pharmaceutically acceptable salt or solvate thereof,

wherein

A is a poly(ADP-ribose) polymerase (PARP) inhibitor moiety;

B is an anti-oxidant moiety selected from radicals (B₁)-(B₆):

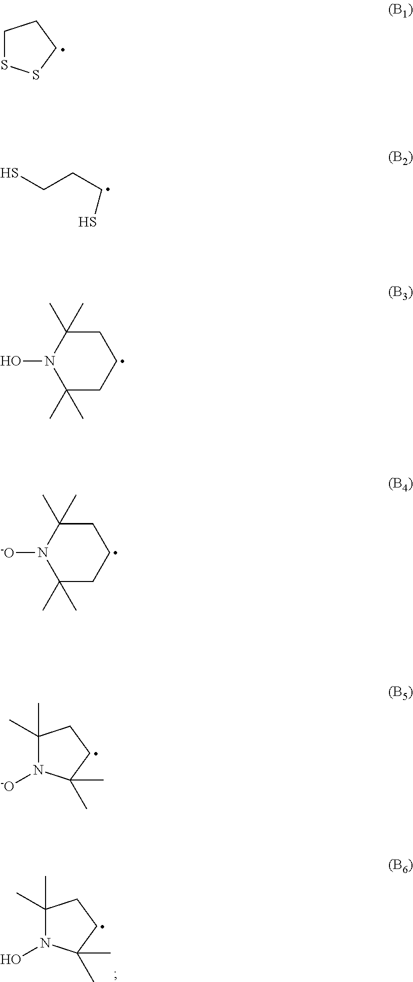

X is a covalent bond or represents one, two or three divalent moieties linked to each other, each independently selected from —O—, —S—, —CO—, —NH—, —NHCONH—, (C₁-C₆)alkylene-, —N—(C₁-C₆)alkylene-, —(C₁-C₆)alkylene-O—CO—(C₁-C₆)alkylene-, —(C₁-C₆)alkylene-O—CO—, —(C₁-C₆)alkylene-NH—CO—(C₁-C₆)alkylene-, —(C₁-C₆)alkylene-NH—CO—, —O—(C₁-C₆)alkylene-, —O—CO—(C₁-C₆)alkylene-, —O—CO—, or a divalent cyclic radical selected from pyrrolidine-diyl, piperidine-diyl, (C₆-C₁₄)arylene-diyl, (C₄-C₁₂)cycloalkane-diyl, or 4-12-membered heterocyclic-diyl, wherein each one of said divalent cyclic radicals may be unsubstituted or substituted with one or more substituents each independently selected from halogen, —OH, —SH, —NH₂, —NO₂, (C₁-C₄)alkyl, —O—(C₁-C₄)alkyl, or —S—(C₁-C₄)alkyl; and

the dot (•) represents the position of attachment to —X-A.

In another aspect, the present invention relates to a pharmaceutical composition comprising a compound of the general formula A-X—B as defined above, or an enantiomer, diastereomer, racemate, or a pharmaceutically acceptable salt or solvate thereof, and a pharmaceutically acceptable carrier.

The compounds and pharmaceutical compositions of the present invention are useful for prevention, treatment, or management of diseases, disorders and conditions associated with elevated PARP activity or expression.

Thus, in a further aspect, the present invention relates to a compound of the general formula A-X—B as defined above, or an enantiomer, diastereomer, racemate, or a pharmaceutically acceptable salt or solvate thereof, for use in prevention, treatment, or management of a disease, disorder or condition associated with elevated PARP activity or expression.

In still a further aspect, the present invention provides a method for prevention, treatment, or management of a disease, disorder or condition associated with elevated PARP activity or expression, said method comprising administering to an individual in need a therapeutically effective amount of a compound of the general formula A-X—B as defined above, or an enantiomer, diastereomer, racemate, or a pharmaceutically acceptable salt or solvate thereof.

### BRIEF DESCRIPTION OF DRAWINGS

FIG. 1 shows the PARP inhibition activity of compound 1 (R-503) evaluated in in vitro potency assay using Universal calorimetric PARP-1 Assay Kit (Trevigen). As shown, compound 1 is a potent PARP inhibitor (IC₅₀=26 nM).

FIG. 2 shows the cytoprotective potency of compound 1 (R-503) as compared to the potent monofunctional PARP inhibitor ABT-888, in RAW cells exposed to H₂O₂ (see Example 6). ABT-888 slightly restored viability (from 65% to 75%), whereas compound 1 markedly increased viability (from 65% to 90%) (p<0.05; compound 1 vs. ABT-888).

FIGS. 3A-3B show the effect of compound 1 (R-503) on the lung myeloperoxidase (MPO) activity (3A) and the lung histology (3B) in a rodent model of inflammation induced by zymosan (see Example 7). As shown, compound 1, administered (60 mg/kg IP) at 1 hour after zymosan challenge (500 mg/kg IP), reduced elevations in lung MPO activity and diminished histological injury by 65% and 80%, respectively (p<0.001 vs. vehicle control).

FIGS. 4A-4B show the effect of compound 1 (R-503) on the lung MPO activity (4A) and the lung histology (4B) in a murine model of chlorine inhalational lung injury (see Example 8). As shown, compound 1 therapy (a q12h regimen of 30 mg/kg/dose IP in 0.5 ml D5W) reduced the elevation in MPO and the elevation in histological lung damage by 82% and 73%, respectively, relative to placebo (p<0.0001).

### DETAILED DESCRIPTION OF THE INVENTION

The present invention provides bifunctional chemical compounds of the general formula A-X—B as defined above, in which two independent chemical moieties, in particular, a poly(ADP-ribose) polymerase (PARP) inhibitor moiety herein designated A and a reactive oxygen species (ROS) scavenger moiety, i.e., a ROS detoxifying group, herein designated B, are covalently attached either directly or via a divalent moiety herein designated X, useful for prevention, treatment, or management of a variety of diseases, disorders or conditions. More particularly, the invention provides PARP inhibitors covalently linked either directly or via a linker to derivatives and analogs of lipoic acid or cyclic nitroxides, such that the resultant molecules have both PARP inhibiting and ROS detoxifying properties.

The PARP inhibitor according to the present invention may be any group capable of inhibiting the activity of the enzyme PARP. The ROS detoxifying group can be any structural derivative or analog of lipoic acid containing the endocyclic disulfide-containing 5- or 6-membered ring, its reduced di-thiol equivalent or complexes thereof. Alternatively, the ROS detoxifying group may be a cyclic nitroxide, preferably a pyrrolidine- or piperidine-based nitroxide or the corresponding reduced hydroxylamine, more preferably 2,2,5,5-tetramethyl pyrrolidine 1-oxide or 2,2,6,6-tetramethylpiperidine 1-oxide.

In certain embodiments, the PARP inhibitor moiety of the present invention is a radical of the formula A₁, A₂ or A₃:

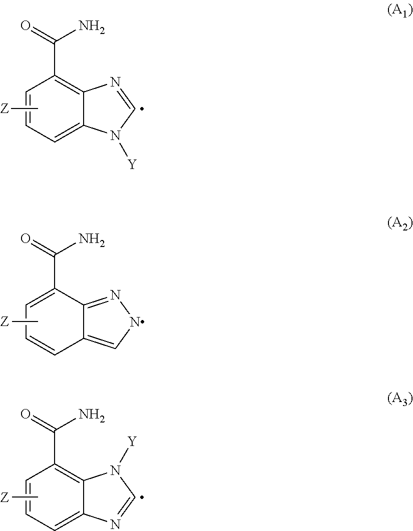

wherein

Y is selected from H, —OH, halogen, —CN, —(C₁-C₆)alkyl, —CO—(C₁-C₆)alkyl, —CO—O—(C₁-C₆)alkyl, —CO—(C₆-C₁₄)aryl, —CO-(4-12-membered heterocyclyl), —(C₃-C₈)monocyclic cycloalkyl, —N(R)₂, —(C₁-C₆)alkylene-N(R)₂, —N(Z)₂, —(C₁-C₆)alkylene-N(Z)₂, —S(O)₂—(C₁-C₆)alkyl, —S(O)₂NH—(C₁-C₆)alkyl, 3-8-membered heterocyclyl, or —(C₁-C₅)alkylene-(3-8-membered heterocyclyl), each of which other than —H, —OH, halogen, and —CN is independently unsubstituted or substituted with one or more substituents each independently selected from halogen, —OH, —N(R)₂, —CF₃, —(C₁-C₆)alkyl, —O—(C₁-C₆)alkyl, —(C₆)aryl optionally substituted with at least one halogen, 3-7-membered heterocyclyl, —(C₁-C₆)alkylene-(C₆)aryl, —(C₁-C₆)alkylene-O—(C₁-C₆)alkyl, —C≡C—(C₁-C₄)alkyl-O—(C₁-C₆)alkyl), —(C₁-C₆)alkylene-OH, —(C₁-C₆)alkylene-N(R)₂, —(C₁-C₆)alkylene-CO—O—(C₁-C₆)alkyl, —CO—O—(C₁-C₆)alkyl, —CO—(C₁-C₆)alkylene-OH, —CO—N(R)₂, or —CO—(C₁-C₆)alkylene-N(R)₂;

R is independently H, (C₁-C₄)alkyl, (C₆)aryl, or 3-7-membered heterocyclyl;

Z is independently H, —OH —CN, —NO₂, halogen, —CH₃, —OCH₃, —CF₃ or —OCF₃; and

the dot (•) represents the position of attachment to —X—B.

Particular such embodiments are those wherein the PARP inhibitor moiety is the radical of the formula A₁, wherein both Y and Z are each H; the radical of the formula A₂, wherein Z is H; or the radical of the formula A₃, wherein both Y and Z are each H.

In other embodiments, the PARP inhibitor moiety of the present invention is a moiety of a compound selected from compounds (A₄)-(A₁₄), which may be bound at any position to —X—B:

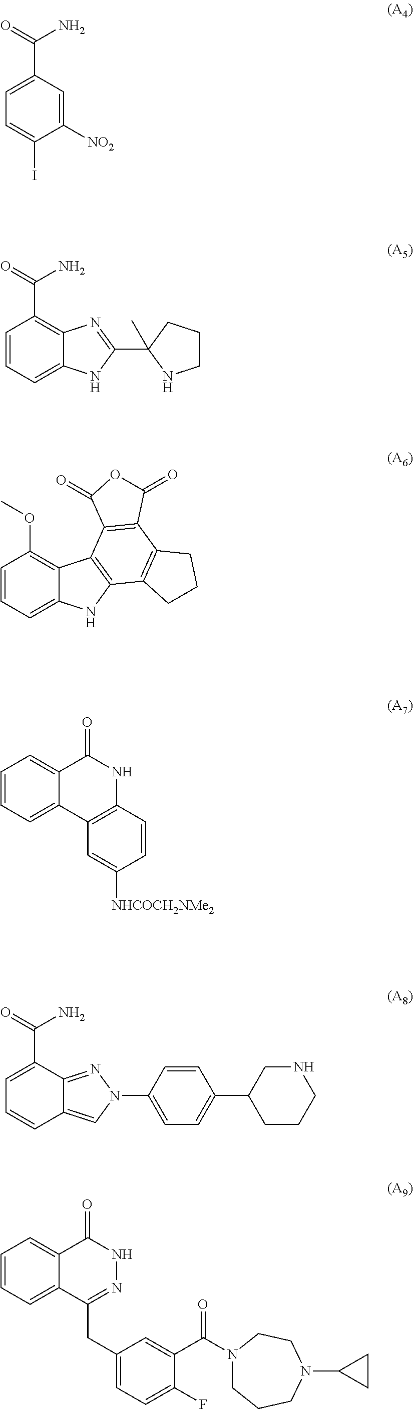

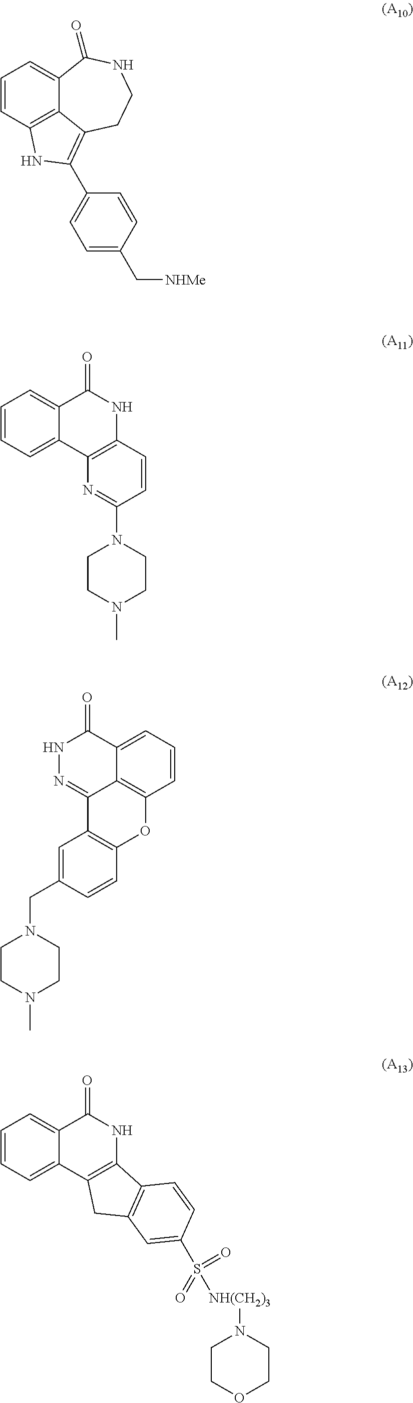

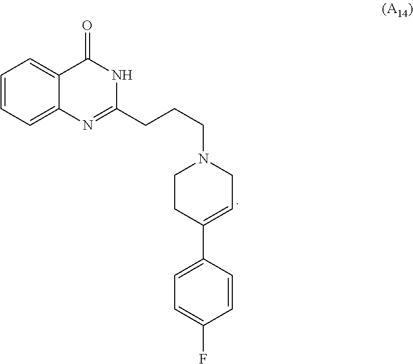 [SMILES: O=c1[nH]c(CCCN2CC=C(c3ccc(F)cc3)CC2)nc2ccccc12]

In further embodiments, the PARP inhibitor moiety of the present invention is selected from benzamide derivatives, benzimidazole derivatives, phthalizinone derivatives, isoindolinone derivatives, phenanthridinone derivatives, or indenoisoquinolinone derivatives.

In still other embodiments, the PARP inhibitor moiety of the present invention is a radical selected from radicals (A₁₅)-(A₂₁):

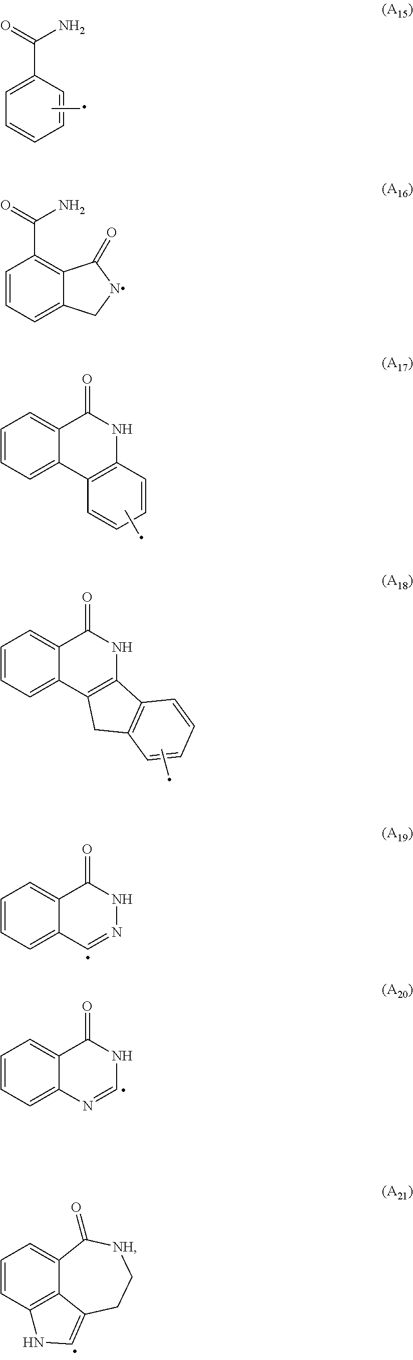

wherein the dot (•) represents the position of attachment to —X—B.

In yet further embodiments, the PARP inhibitor moiety of the present invention is selected from the PARP inhibitors disclosed in U.S. Pat. Nos. 7,041,675, 6,903,098, 6,737,421, 7,456,178, 6,635,642, 7,157,452, 7,235,557, 6,723,733, 6,716,828, 6,545,011, 6,197,785, 6,380,193, 6,395,749, 7,449,464, 7,470,688, 6,664,269, 7,151,102, 7,196,085, 7,407,957, 7,652,028, 7,393,955, 7,268,143, 6,956,035, 6,828,319, 6,534,651, 6,277,990, 7,268,126, 7,547,714, and 7,598,231; US Publication Nos. 2006/0276497, 2007/0179136, 2006/0229289, 2009/0209520, 2007/0093489, 2008/0200469, and 2007/281948A1; and International Publication Nos. WO 2007/149451, WO 2006/110816, WO 2007/113596, WO 2007/138351, WO 2007/144652, WO 2007/144639, and WO 2007/144637, all these patents and publications being hereby incorporated by reference in their entirety as if fully disclosed herein (see also Ferraris, 2010).

As used herein, the term “halogen” includes fluoro, chloro, bromo, and iodo, and is preferably fluoro or chloro.

The term “(C₁-C₆)alkyl” typically means a straight or branched saturated hydrocarbyl having 1-6 carbon atoms and includes, e.g., methyl, ethyl, n-propyl, isopropyl, n-butyl, sec-butyl, isobutyl, tert-butyl, n-pentyl, 2,2-dimethylpropyl, n-hexyl, and the like. Preferred are (C₁-C₄)alkyl groups, most preferably methyl, ethyl and propyl. The term “(C₁-C₆)alkylene” typically means a divalent straight or branched hydrocarbyl radical having 1-6 carbon atoms and includes, e.g., methylene, ethylene, propylene, butylene, 2-methylpropylene, pentylene, 2-methylbutylene, hexylene, 2-methylpentylene, 3-methylpentylene, 2,3-dimethylbutylene, and the like.

The term “(C₆-C₁₄)aryl” denotes an aromatic carbocyclic group having 6 to 14 carbon atoms consisting of a single ring or multiple rings either condensed or linked by a covalent bond such as, but not limited to, phenyl, naphthyl, phenanthryl, and biphenyl, and the term “(C₆)aryl” specifically denotes phenyl. The term “(C₆-C₁₄)arylene-diyl” denotes a divalent aromatic carbocyclic group having 6-14 carbon atoms consisting of a single ring or multiple rings either condensed or linked by a covalent bond such as, but not limited to, phenylene and naphthylene.

The term “(C₃-C₈)monocyclic cycloalkyl” means a cyclic saturated hydrocarbyl group such as cyclopropyl, cyclobutyl, cyclopentyl, cyclohexyl, cycloheptyl, or cyclooctyl.

The term “(C₄-C₁₂)cycloalkane-diyl” means a divalent radical derived from a mono-, bi- or tricyclic ring having 4-12 carbon atoms.

The term “4-12-membered heterocyclic-diyl” as used herein refers to a divalent radical of mono- or poly-cyclic ring of 4-12 atoms containing at least one carbon atom and at least one, preferably 1-2, heteroatoms selected from sulfur, oxygen or nitrogen, that may be saturated or unsaturated, i.e., containing at least one unsaturated bond. Non-limiting examples of such groups include pyridine-diyl, pyrimidine-diyl, dioxane-diyl, pyrrolidine-diyl, piperidine-diyl, and morpholine-diyl. The term “heterocyclyl” as used herein refers to any univalent radical derived from a heterocyclic ring by removal of hydrogen from any ring atom.

The term “pyrrolidine-diyl” encompasses any divalent moiety of pyrrolidine, such as 2,2-pyrrolidine-diyl, 2,3-pyrrolidine-diyl, 2,4-pyrrolidine-diyl, 2,5-pyrrolidine-diyl, and the like.

The term “piperidine-diyl” encompasses any divalent moiety of piperidine, such as 2,4-piperidine-diyl, 2,5-piperidine-diyl, 2,6-piperidine-diyl, and the like.

In certain embodiments, the compound of the present invention is a compound of the general formula A-X—B, wherein X represents one divalent moiety as defined above. Particular compounds are those wherein X is —O—, —S—, —CO—, —NH—, —NHCONH—, —(C₁-C₆)alkylene-, —N—(C₁-C₆)alkylene-, —(C₁-C₆)alkylene-O—CO—(C₁-C₆)alkylene-, —(C₁-C₆)alkylene-O—CO—, —(C₁-C₆)alkylene-NH—CO—(C₁-C₆)alkylene-, —(C₁-C₆)alkylene-NH—CO—, —O—(C₁-C₆)alkylene-, —O—CO—(C₁-C₆)alkylene- or —O—CO—. In certain particular compounds, X represents a straight —(C₁-C₆)alkylene-such as methylene, ethylene, propylene, butylene, pentylene, and hexylene.

In other embodiments, the compound of the present invention is a compound of the general formula A-X—B, wherein X represents two divalent moieties linked to each other —Xₐ—X_{b}—. Particular compounds are those wherein Xₐ is selected from pyrrolidine-diyl, piperidine-diyl, (C₆-C₁₄)arylene-diyl, (C₄-C₁₂)cycloalkane-diyl or 4-12-membered heterocyclic-diyl, optionally substituted with one or more substituents each independently selected from halogen, —OH, —SH, —NH₂, —NO₂, (C₁-C₄)alkyl, —O—(C₁-C₄)alkyl or —S—(C₁-C₄)alkyl; and X_{b} is —(C₁-C₆)alkylene-, —N—(C₁-C₆)alkylene-, —(C₁-C₆)alkylene-O—CO—(C₁-C₆)alkylene-, —(C₁-C₆)alkylene-O—CO—, —(C₁-C₆)alkylene-NH—CO—(C₁-C₆)alkylene-, —(C₁-C₆)alkylene-NH—CO—, —O—(C₁-C₆)alkylene-, —O—CO—(C₁-C₆)alkylene-, or —O—CO—. In certain particular compounds, Xₐ is a pyrrolidine-diyl such as 2,2-pyrrolidine-diyl, 2,3-pyrrolidine-diyl and 2,4-pyrrolidine-diyl; and X_{b} is —(C₁-C₆)alkylene-, —(C₁-C₆)alkylene-O—CO—(C₁-C₆)alkylene-, —O—(C₁-C₆)alkylene-, —O—CO—(C₁-C₆)alkylene-, or —O—CO—.

In further embodiments, the compound of the present invention is a compound of the general formula A-X—B, wherein X represents three divalent moieties linked to each other —Xₐ—X_{b}—X_{c}—. Particular compounds are those wherein Xₐ is selected from pyrrolidine-diyl, piperidine-diyl, (C₆-C₁₄)arylene-diyl, (C₄-C₁₂)cycloalkane-diyl or 4-12-membered heterocyclic-diyl, optionally substituted with one or more substituents each independently selected from halogen, —OH, —SH, —NH₂, —NO₂, (C₁-C₄)alkyl, —O—(C₁-C₄)alkyl, or —S—(C₁-C₄)alkyl; X_{b} is selected from pyrrolidine-diyl, piperidine-diyl, (C₆-C₁₄)arylene-diyl, (C₄-C₁₂)cycloalkane-diyl or 4-12-membered heterocyclic-diyl, optionally substituted with one or more substituents each independently selected from halogen, —OH, —SH, —NH₂, —NO₂, (C₁-C₄)alkyl, —O—(C₁-C₄)alkyl, or —S—(C₁-C₄)alkyl; and X_{b} is —(C₁-C₆)alkylene-, —N—(C₁-C₆)alkylene-, —(C₁-C₆)alkylene-O—CO—(C₁-C₆)alkylene-, —(C₁-C₆)alkylene-O—CO—, —(C₁-C₆)alkylene-NH—CO—(C₁-C₆)alkylene-, —(C₁-C₆)alkylene-NH—CO—, —O—(C₁-C₆)alkylene-, —O—CO—(C₁-C₆)alkylene-, or —O—CO—. In certain particular compounds, Xₐ is (C₆-C₁₄)arylene-diyl, e.g., (C₆)arylene such as 1,4-phenylene and the like, optionally substituted with halogen, such as 3-fluoro-1,4 phenylene and 3-chloro-1,4 phenylene; X_{b} is a piperidine-diyl such as 2,6-piperidine-diyl; and X_{c} is —(C₁-C₆)alkylene-O—CO—(C₁-C₆)alkylene-, —(C₁-C₆)alkylene-O—CO—, —(C₁-C₆)alkylene-NH—CO—(C₁-C₆)alkylene-, or —(C₁-C₆)alkylene-NH—CO—.

The specific divalent moieties X according to the general formula A-X—B, which are described in the specification are herein identified as linkers X₁ to X₁₇, and their full chemical structures are depicted in Table 1 hereinafter.

TABLE 1
Divalent linkers (X) used in certain
compounds of the invention

| Linker | Structure• |
|---|---|
| X₁ | —(CH₂)₄— |
| X₂ | —(CH₂)₅— |
| X₃ | 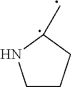 |
| X₄ | 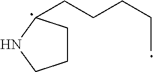 |
| X₅ | 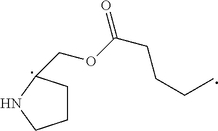 |
| X₆ | 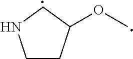 |
| X₇ | 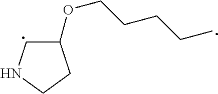 |
| X₈ | 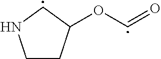 |
| X₉ | 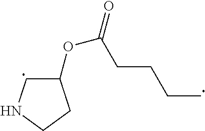 |
| X₁₀ | 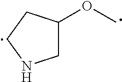 |
| X₁₁ | 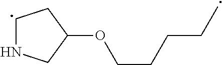 |
| X₁₂ | 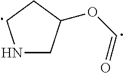 |
| X₁₃ | 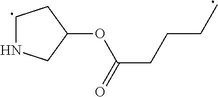 |
| X₁₄ | 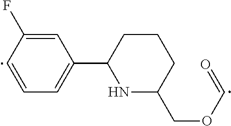 |
| X₁₅ | 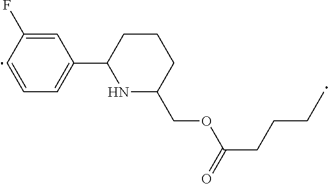 |
| X₁₆ | 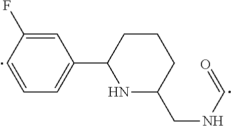 |
| X₁₇ | 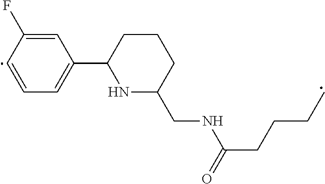 |

•The left and right dots (•) represent the positions of attachment to A and B moieties, respectively.

In particular embodiments, the compound of the present invention is a compound of the general formula A-X—B, wherein X represents one divalent moiety selected from —(CH₂)₄— or —(CH₂)₅— (linkers X₁ and X₂, respectively).

In other particular embodiments, the compound of the present invention is a compound of the general formula A-X—B, wherein X represents two divalent moieties linked to each other —Xₐ—X_{b}—, wherein Xₐ is 2,2-pyrrolidine-diyl; and X_{b} is —CH₂—, —(CH₂)₅— or —CH₂—O—CO—(CH₂)₄—, linked at position 2 of the 2,2-pyrrolidine-diyl (linkers X₃, X₄ and X₅, respectively).

In further particular embodiments, the compound of the present invention is a compound of the general formula A-X—B, wherein X represents two divalent moieties linked to each other —Xₐ—X_{b}—, wherein Xₐ is 2,3-pyrrolidine-diyl; and X_{b} is selected from —O—CH₂—, —O—(CH₂)₅—, —O—CO— or —O—CO—(CH₂)₄—, linked at position 3 of the 2,3-pyrrolidine-diyl (linkers X₆, X₇, X₈ and X₉, respectively).

In still other particular embodiments, the compound of the present invention is a compound of the general formula A-X—B, wherein X represents two divalent moieties linked to each other —Xₐ—X_{b}—, wherein Xₐ is 2,4-pyrrolidine-diyl; and X_{b} is selected from —O—CH₂—, —O—(CH₂)₅—, —O—CO— or —O—CO—(CH₂)₄—, linked at position 4 of the 2,4-pyrrolidine-diyl (linkers X₁₀, X₁₁, X₁₂ and X₁₃, respectively).

In yet other particular embodiments, the compound of the present invention is a compound of the general formula A-X—B, wherein X represents three divalent moieties linked to each other —Xₐ—X_{b}—X_{c}—, wherein Xₐ is 3-fluoro-1,4 phenylene; X_{b} is 2,6-piperidine-diyl linked at position 1 of the 3-fluoro-1,4 phenylene; and X, is selected from —CH₂—O—CO—, —CH₂—O—CO—(CH₂)₄—, —CH₂—NH—CO— or —CH₂—NH—CO—(CH₂)₄—, linked at position 6 of the 2,6-piperidine-diyl (linkers X₁₄, X₁₅, X₁₆ and X₁₇, respectively).

The specific compounds of the general formula A-X—B described in the specification are herein identified by the Arabic numbers 1-26 in bold (compound 1 is also identified R-503), and their full chemical structures are depicted in Table 2.

In certain embodiments, the compound of the present invention is a compound of the general formula A-X—B as defined above, wherein (i) A is radical A₁ and B is radical B₁; (ii) A is radical A₁ and B is radical B₅; (iii) A is radical A₁ and B is radical B₄; (iv) A is radical A₂ and B is radical B₁; or (v) A is radical A₂ and B is radical B₅.

In one specific embodiment, the compound of the invention is a compound of the general formula A-X—B, wherein A is radical A₁ wherein both Y and Z are H; B is radical B₁; and X is —(CH₂)₄— (linker X₁), i.e., 2-(4-(1,2-dithiolan-3-yl)butyl)-1H-benzo[d]imidazole-4-carboxamide (compound 1; R-503).

In another specific embodiment, the compound of the invention is a compound of the general formula A-X—B, wherein A is radical A₂ wherein Z is H; B is radical B₁; and X is —(CH₂)₅— (linker X₂), i.e., 2-(5-(1,2-dithiolan-3-yl)pentyl)-2H-indazole-7-carboxamide (compound 2).

In a further specific embodiment, the compound of the invention is a compound of the general formula A-X—B, wherein A is radical A₁ wherein both Y and Z are H; B is radical B₁; and X represents two divalent moieties linked to each other —Xₐ—X_{b}—, wherein Xₐ is 2,2-pyrrolidine-diyl; and X_{b} is —(CH₂)₅— (linker X₄), i.e., 2-(2-(5-(1,2-dithiolan-3-yl)pentyl)pyrrolidin-2-yl)-1H-benzo[d]imidazole-4-carboxamide (compound 3).

In still a further specific embodiment, the compound of the invention is a compound of the general formula A-X—B, wherein A is radical A₁ wherein both Y and Z are H; B is radical B₁; and X represents two divalent moieties linked to each other —Xₐ—X_{b}—, wherein Xₐ is 2,2-pyrrolidine-diyl; and X_{b} is —CH₂—O—C(O)—(CH₂)₄-(linker X₅), i.e., (2-(4-carbamoyl-1H-benzo[d]imidazol-2-yl)pyrrolidin-2-yl)methyl 5-(1,2-dithiolan-3-yl)pentanoate (compound 4).

In yet a further specific embodiment, the compound of the invention is a compound of the general formula A-X—B, wherein A is radical A₁ wherein both Y and Z are H; B is radical B₁; and X represents two divalent moieties linked to each other —Xₐ—X_{b}—, wherein Xₐ is 2,3-pyrrolidine-diyl; and X_{b} is —O—(CH₂)₅— linked at position 3 of the 2,3-pyrrolidine-diyl (linker X₇), i.e., 2-(3-(5-(1,2-dithiolan-3-yl) pentyloxy)pyrrolidin-2-yl)-1H-benzo[d]imidazole-4-carboxamide (compound 5).

In still another specific embodiment, the compound of the invention is a compound of the general formula A-X—B, wherein A is radical A₁ wherein both Y and Z are H; B is radical B₁; and X represents two divalent moieties linked to each other —Xₐ—X_{b}—, wherein Xₐ is 2,3-pyrrolidine-diyl; and X_{b} is —O—C(O)—(CH₂)₄— linked at position 3 of the 2,3-pyrrolidine-diyl (linker X₉), i.e., 2-(4-carbamoyl-1H-benzo[d]imidazol-2-yl)pyrrolidin-3-yl-5-(1,2-dithiolan-3-yl)pentanoate (compound 6).

In yet another specific embodiment, the compound of the invention is a compound of the general formula A-X—B, wherein A is radical A₁ wherein both Y and Z are H; B is radical B₁; and X represents two divalent moieties linked to each other —Xₐ—X_{b}—, wherein Xₐ is 2,4-pyrrolidine-diyl; and X_{b} is —O—(CH₂)₅— linked at position 4 of the 2,4-pyrrolidine-diyl (linker X₁₁), i.e., 2-(4-(5-(1,2-dithiolan-3-yl)pentyloxy)pyrrolidin-2-yl)-1H-benzo[d]imidazole-4-carboxamide (compound 7).

In still a further specific embodiment, the compound of the invention is a compound of the general formula A-X—B, wherein A is radical A₁ wherein both Y and Z are H; B is radical B₁; and X represents two divalent moieties linked to each other —Xₐ—X_{b}—, wherein Xₐ is 2,4-pyrrolidine-diyl; and X_{b} is —O—C(O)—(CH₂)₄— linked at position 4 of the 2,4-pyrrolidine-diyl (linker X₁₃), i.e., 5-(4-carbamoyl-1H-benzo[d]imidazol-2-yl)pyrrolidin-3-yl 5-(1,2-dithiolan-3-yl)pentanoate (compound 8).

In yet a further specific embodiment, the compound of the invention is a compound of the general formula A-X—B, wherein A is radical A₁ wherein both Y and Z are H; B is radical B₅; and X represents two divalent moieties linked to each other —Xₐ—X_{b}—, wherein Xₐ is 2,2-pyrrolidine-diyl; and X_{b} is —CH₂— (linker X₃), i.e., 3-((2-(4-carbamoyl-1H-benzo[d]imidazol-2-yl)pyrrolidin-2-yl)methyl)-2,2,5,5-tetramethylpyrrolidin-1-olate (compound 9).

In still another specific embodiment, the compound of the invention is a compound of the general formula A-X—B, wherein A is radical A₁ wherein both Y and Z are H; B is radical B₅; and X represents two divalent moieties linked to each other —Xₐ—X_{b}—, wherein Xₐ is 2,3-pyrrolidine-diyl; and X_{b} is —O—CH₂— linked at position 3 of the 2,3-pyrrolidine-diyl (linker X₆), i.e., 3-((2-(4-carbamoyl-1H-benzo[d]imidazol-2-yl)pyrrolidin-3-yloxy)methyl)-2,2,5,5-tetramethylpyrrolidin-1-olate (compound 10).

In yet another specific embodiment, the compound of the invention is a compound of the general formula A-X—B, wherein A is radical A₁ wherein both Y and Z are H; B is radical B₅; and X represents two divalent moieties linked to each other —Xₐ—X_{b}—, wherein Xₐ is 2,3-pyrrolidine-diyl; and X_{b} is —O—C(O)— linked at position 3 of the 2,3-pyrrolidine-diyl (linker X₈), i.e., 3-((2-(4-carbamoyl-1H-benzo[d]imidazol-2-yl)pyrrolidin-3-yloxy)carbonyl)-2,2,5,5-tetramethylpyrrolidin-1-olate (compound 11).

In still a further specific embodiment, the compound of the invention is a compound of the general formula A-X—B, wherein A is radical A₁ wherein both Y and Z are H; B is radical B₅; and X represents two divalent moieties linked to each other —Xₐ—X_{b}—, wherein Xₐ is 2,4-pyrrolidine-diyl; and X_{b} is —O—CH₂— linked at position 4 of the 2,4-pyrrolidine-diyl (linker X₁₀), i.e., 3-((2-(4-carbamoyl-1H-benzo[d]imidazol-2-yl)pyrrolidin-3-yloxy)methyl)-2,2,5,5-tetramethylpyrrolidin-1-olate (compound 12).

In yet a further specific embodiment, the compound of the invention is a compound of the general formula A-X—B, wherein A is radical A₁ wherein both Y and Z are H; B is radical B₅; and X represents two divalent moieties linked to each other —Xₐ—X_{b}—, wherein Xₐ is 2,4-pyrrolidine-diyl; and X_{b} is —O—C(O)— linked at position 4 of the 2,4-pyrrolidine-diyl (linker X₁₂), i.e., 345-(4-carbamoyl-1H-benzo[d]imidazol-2-yl)pyrrolidin-3-yloxy)carbonyl)-2,2,5,5-tetramethylpyrrolidin-1-olate (compound 13).

In still another specific embodiment, the compound of the invention is a compound of the general formula A-X—B, wherein A is radical A₁ wherein both Y and Z are H; B is radical B₄; and X represents two divalent moieties linked to each other —Xₐ—X_{b}—, wherein Xₐ is 2,2-pyrrolidine-diyl; and X_{b} is —CH₂— (linker X₃), i.e., 4-((2-(4-carbamoyl-1H-benzo[d]imidazol-2-yl)pyrrolidin-2-yl)methyl)-2,2,6,6-tetramethylpiperidin-1-olate (compound 14).

In yet another specific embodiment, the compound of the invention is a compound of the general formula A-X—B, wherein A is radical A₁ wherein both Y and Z are H; B is radical B₄; and X represents two divalent moieties linked to each other —Xₐ—X_{b}—, wherein Xₐ is 2,3-pyrrolidine-diyl; and X_{b} is —O—CH₂— linked at position 3 of the 2,3-pyrrolidine-diyl (linker X₆), i.e., 4-((2-(4-carbamoyl-1H-benzo[d]imidazol-2-yl)pyrrolidin-3-yloxy)methyl)-2,2,6,6-tetramethylpiperidin-1-olate (compound 15).

In still a further specific embodiment, the compound of the invention is a compound of the general formula A-X—B, wherein A is radical A₁ wherein both Y and Z are H; B is radical B₄; and X represents two divalent moieties linked to each other —Xₐ—X_{b}—, wherein Xₐ is 2,3-pyrrolidine-diyl; and X_{b} is —O—C(O)— linked at position 3 of the 2,3-pyrrolidine-diyl (linker X₈), i.e., 4-((2-(4-carbamoyl-1H-benzo[d]imidazol-2-yl)pyrrolidin-3-yloxy)carbonyl)-2,2,6,6-tetramethyl piperidin-1-olate (compound 16).

In yet a further specific embodiment, the compound of the invention is a compound of the general formula A-X—B, wherein A is radical A₁ wherein both Y and Z are H; B is radical B₄; and X represents two divalent moieties linked to each other —Xₐ—X_{b}—, wherein Xₐ is 2,4-pyrrolidine-diyl; and X_{b} is —O—CH₂— linked at position 4 of the 2,4-pyrrolidine-diyl (linker X₁₀), i.e., 4-((5-(4-carbamoyl-1H-benzo[d]imidazol-2-yl)pyrrolidin-3-yloxy)methyl)-2,2,6,6-tetramethylpiperidin-1-olate (compound 17).

In still another specific embodiment, the compound of the invention is a compound of the general formula A-X—B, wherein A is radical A₁ wherein both Y and Z are H; B is radical B₄; and X represents two divalent moieties linked to each other —Xₐ—X_{b}—, wherein Xₐ is 2,4-pyrrolidine-diyl; and X_{b} is —O—C(O)— linked at position 4 of the 2,4-pyrrolidine-diyl (linker X₁₂), i.e., 4-((5-(4-carbamoyl-1H-benzo[d]imidazol-2-yl)pyrrolidin-3-yloxy)carbonyl)-2,2,6,6-tetramethyl piperidin-1-olate (compound 18).

In yet another specific embodiment, the compound of the invention is a compound of the general formula A-X—B, wherein A is radical A₁ wherein both Y and Z are H; B is radical B₁; and X represents three divalent moieties linked to each other —Xₐ—X_{b}—X_{c}—, wherein Xₐ is 3-fluoro-1,4 phenylene; X_{b} is 2,6-piperidine-diyl linked at position 1 of the 3-fluoro-1,4 phenylene; and X, is —CH₂—O—C(O)—(CH₂)₄-linked at position 6 of the 2,6-piperidine-diyl (linker X₁₅), i.e., (6-(4-(4-carbamoyl-1-benzo[d]imidazol-2-yl)-3-fluorophenyl)piperidin-2-yl)methyl 5-(1,2-dithiolan-3-yl)pentanoate (compound 19).

In still a further specific embodiment, the compound of the invention is a compound of the general formula A-X—B, wherein A is radical A₁ wherein both Y and Z are H; B is radical B₁; and X represents three divalent moieties linked to each other —Xₐ—X_{b}—X_{c}—, wherein Xₐ is 3-fluoro-1,4 phenylene; X_{b} is 2,6-piperidine-diyl linked at position 1 of the 3-fluoro-1,4 phenylene; and X_{c} is —CH₂—NH—C(O)—(CH₂)₄-linked at position 6 of the 2,6-piperidine-diyl (linker X₁₇), i.e., 2-(4-(6-((5-(1,2-dithiolan-3-yl)pentanamido)methyl)piperidin-2-yl)-2-fluorophenyl)-1H-benzo[d]imidazole-4-carboxamide (compound 20).

In yet a further specific embodiment, the compound of the invention is a compound of the general formula A-X—B, wherein A is radical A₁ wherein both Y and Z are H; B is radical B₅; and X represents three divalent moieties linked to each other —X, X_{b}—X_{c}—, wherein Xₐ is 3-fluoro-1,4 phenylene; X_{b} is 2,6-piperidine-diyl linked at position 1 of the 3-fluoro-1,4 phenylene; and X_{c} is —CH₂—O—C(O)— linked at position 6 of the 2,6-piperidine-diyl (linker X₁₄), i.e., 3-(((6-(4-(4-carbamoyl-1H-benzo[d]imidazol-2-yl)-3-fluorophenyl)piperidin-2-yl)methoxy)carbonyl)-2,2,5,5-tetramethylpyrrolidin-1-olate (compound 21).

In still another specific embodiment, the compound of the invention is a compound of the general formula A-X—B, wherein A is radical A₁ wherein both Y and Z are H; B is radical B₅; and X represents three divalent moieties linked to each other —X, X_{b}—X_{c}—, wherein Xₐ is 3-fluoro-1,4 phenylene; X_{b} is 2,6-piperidine-diyl linked at position 1 of the 3-fluoro-1,4 phenylene; and X_{c} is —CH₂—NH—C(O)— linked at position 6 of the 2,6-piperidine-diyl (linker X₁₆), i.e., 3-((6-(4-(4-carbamoyl-1H-benzo[d]imidazol-2-yl)-3-fluorophenyl)piperidin-2-yl)methylcarbamoyl)-2,2,5,5-tetramethylpyrrolidin-1-olate (compound 22).

In yet another specific embodiment, the compound of the invention is a compound of the general formula A-X—B, wherein A is radical A₂ wherein Z is H; B is radical B₁; and X represents three divalent moieties linked to each other —Xₐ—X_{b}—X_{c}—, wherein Xₐ is 3-fluoro-1,4 phenylene; X_{b} is 2,6-piperidine-diyl linked at position 1 of the 3-fluoro-1,4 phenylene; and X_{b} is —CH₂—O—C(O)—(CH₂)₄— linked at position 6 of the 2,6-piperidine-diyl (linker X₁₅), i.e., (6-(4-(7-carbamoyl-2H-indazol-2-yl)-3-fluorophenyl)piperidin-2-yl)methyl 5-(1,2-dithiolan-3-yl)pentanoate (compound 23).

In still a further specific embodiment, the compound of the invention is a compound of the general formula A-X—B, wherein A is radical A₂ wherein Z is H; B is radical B₁; and X represents three divalent moieties linked to each other —Xₐ—X_{b}—X_{c}—, wherein Xₐ is 3-fluoro-1,4 phenylene; X_{b} is 2,6-piperidine-diyl linked at position 1 of the 3-fluoro-1,4 phenylene; and X_{b} is —CH₂—NH—C(O)—(CH₂)₄— linked at position 6 of the 2,6-piperidine-diyl (linker X₁₇), i.e., 2-(4-(6-((5-(1,2-dithiolan-3-yl)pentanamido)methyl)piperidin-2-yl)-2-fluorophenyl)-2H-indazole-7-carboxamide (compound 24).

In yet a further specific embodiment, the compound of the invention is a compound of the general formula A-X—B, wherein A is radical A₂ wherein Z is H; B is radical B₅; and X represents three divalent moieties linked to each other wherein Xₐ is 3-fluoro-1,4 phenylene; X_{b} is 2,6-piperidine-diyl linked at position 1 of the 3-fluoro-1,4 phenylene; and X_{b} is —CH₂—O—C(O)— linked at position 6 of the 2,6-piperidine-diyl (linker X₁₄), i.e., 3-(((6-(4-(7-carbamoyl-2H-indazol-2-yl)-3-fluorophenyl)piperidin-2-yl)methoxy)carbonyl)-2,2,5,5-tetramethylpyrrolidin-1-olate (compound 25).

In still another specific embodiment, the compound of the invention is a compound of the general formula A-X—B, wherein A is radical A₂ wherein Z is H; B is radical B₅; and X represents three divalent moieties linked to each other —Xₐ—X_{b}—X_{c}—, wherein Xₐ is 3-fluoro-1,4 phenylene; X_{b} is 2,6-piperidine-diyl linked at position 1 of the 3-fluoro-1,4 phenylene; and X, is —CH₂—NH—C(O)— linked at position 6 of the 2,6-piperidine-diyl (linker X₁₆), i.e., 3-((6-(4-(7-carbamoyl-2H-indazol-2-yl)-3-fluorophenyl)piperidin-2-yl)methylcarbamoyl)-2,2,5,5-tetramethylpyrrolidin-1-olate (compound 26).

TABLE 2
Specific compounds of the invention listed in the specification

| No. | Structure |
|---|---|
| 1 | 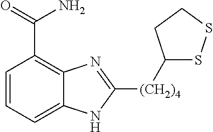 |
| 2 | 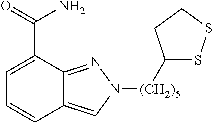 |
| 3 | 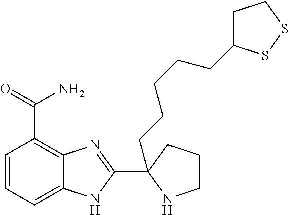 [SMILES: NC(=O)c1cccc2[nH]c(C3(CCCCCC4CCSS4)CCCN3)nc12] |
| 4 | 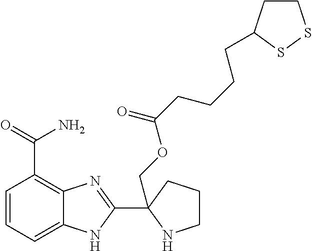 [SMILES: NC(=O)c1cccc2[nH]c(C3(COC(=O)CCCCC4CCSS4)CCCN3)nc12] |
| 5 | 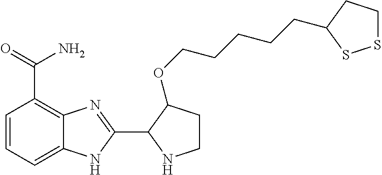 [SMILES: NC(=O)c1cccc2[nH]c(C3NCCC3OCCCCCC3CCSS3)nc12] |
| 6 | 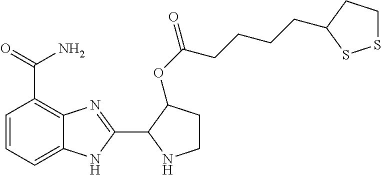 [SMILES: NC(=O)c1cccc2[nH]c(C3NCCC3OC(=O)CCCCC3CCSS3)nc12] |
| 7 | 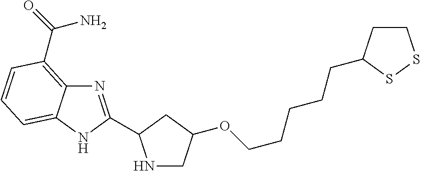 [SMILES: NC(=O)c1cccc2[nH]c(C3CC(OCCCCCC4CCSS4)CN3)nc12] |
| 8 | 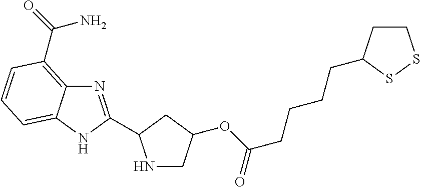 [SMILES: NC(=O)c1cccc2[nH]c(C3CC(OC(=O)CCCCC4CCSS4)CN3)nc12] |
| 9 | 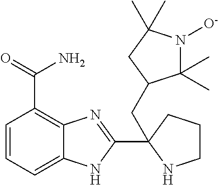 [SMILES: CC1(C)CC(CC2(c3nc4c(C(N)=O)cccc4[nH]3)CCCN2)C(C)(C)N1[O-]] |
| 10 | 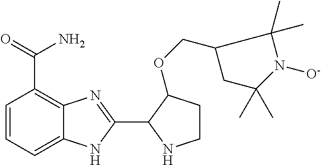 [SMILES: CC1(C)CC(COC2CCNC2c2nc3c(C(N)=O)cccc3[nH]2)C(C)(C)N1[O-]] |
| 11 | 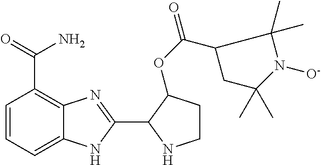 [SMILES: CC1(C)CC(C(=O)OC2CCNC2c2nc3c(C(N)=O)cccc3[nH]2)C(C)(C)N1[O-]] |
| 12 | 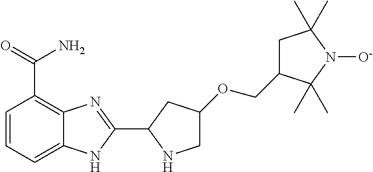 [SMILES: CC1(C)CC(COC2CNC(c3nc4c(C(N)=O)cccc4[nH]3)C2)C(C)(C)N1[O-]] |
| 13 | 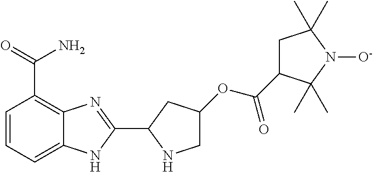 [SMILES: CC1(C)CC(C(=O)OC2CNC(c3nc4c(C(N)=O)cccc4[nH]3)C2)C(C)(C)N1[O-]] |
| 14 | 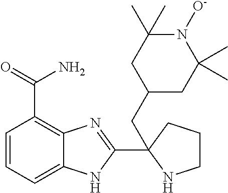 [SMILES: CC1(C)CC(CC2(c3nc4c(C(N)=O)cccc4[nH]3)CCCN2)CC(C)(C)N1[O-]] |
| 15 | 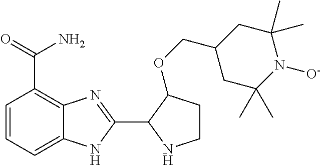 [SMILES: CC1(C)CC(COC2CCNC2c2nc3c(C(N)=O)cccc3[nH]2)CC(C)(C)N1[O-]] |
| 16 | 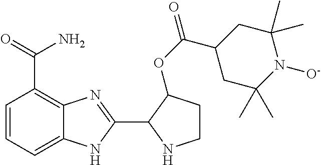 [SMILES: CC1(C)CC(C(=O)OC2CCNC2c2nc3c(C(N)=O)cccc3[nH]2)CC(C)(C)N1[O-]] |
| 17 | 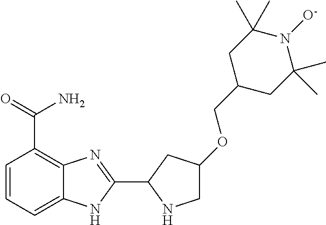 [SMILES: CC1(C)CC(COC2CNC(c3nc4c(C(N)=O)cccc4[nH]3)C2)CC(C)(C)N1[O-]] |
| 18 | 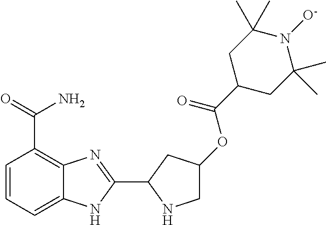 [SMILES: CC1(C)CC(C(=O)OC2CNC(c3nc4c(C(N)=O)cccc4[nH]3)C2)CC(C)(C)N1[O-]] |
| 19 | 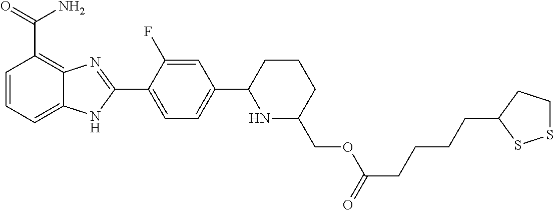 [SMILES: NC(=O)c1cccc2[nH]c(-c3ccc(C4CCCC(COC(=O)CCCCC5CCSS5)N4)cc3F)nc12] |
| 20 | 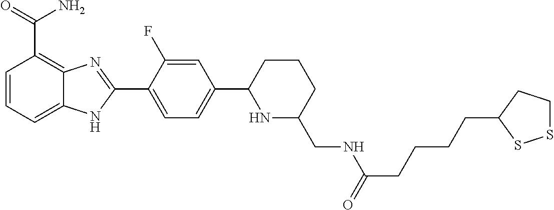 [SMILES: NC(=O)c1cccc2[nH]c(-c3ccc(C4CCCC(CNC(=O)CCCCC5CCSS5)N4)cc3F)nc12] |
| 21 | 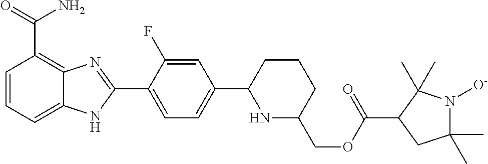 [SMILES: CC1(C)CC(C(=O)OCC2CCCC(c3ccc(-c4nc5c(C(N)=O)cccc5[nH]4)c(F)c3)N2)C(C)(C)N1[O-]] |
| 22 | 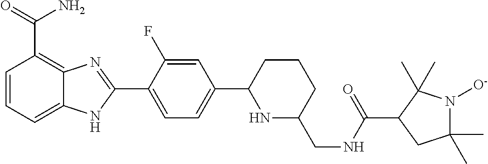 [SMILES: CC1(C)CC(C(=O)NCC2CCCC(c3ccc(-c4nc5c(C(N)=O)cccc5[nH]4)c(F)c3)N2)C(C)(C)N1[O-]] |
| 23 | 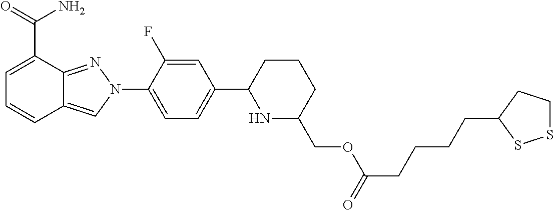 [SMILES: NC(=O)c1cccc2cn(-c3ccc(C4CCCC(COC(=O)CCCCC5CCSS5)N4)cc3F)nc12] |
| 24 | 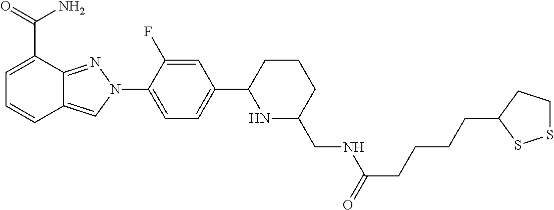 [SMILES: NC(=O)c1cccc2cn(-c3ccc(C4CCCC(CNC(=O)CCCCC5CCSS5)N4)cc3F)nc12] |
| 25 | 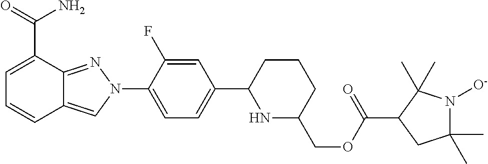 [SMILES: CC1(C)CC(C(=O)OCC2CCCC(c3ccc(-n4cc5cccc(C(N)=O)c5n4)c(F)c3)N2)C(C)(C)N1[O-]] |
| 26 | 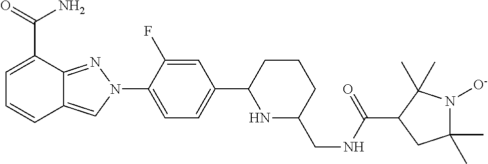 [SMILES: CC1(C)CC(C(=O)NCC2CCCC(c3ccc(-n4cc5cccc(C(N)=O)c5n4)c(F)c3)N2)C(C)(C)N1[O-]] |

The compounds of the present invention may have one or more asymmetric centers, and may accordingly exist both as enantiomers (R, S, or racemate) and as diastereoisomers. Specifically, those chiral centers may be in either or both the PARP inhibitor moiety A and the anti-oxidant moiety B, as well as in the linker X in cases wherein X represents two or three divalent moieties linked to each other. It should be understood that the present invention encompasses all such enantiomers, isomers and mixtures thereof, as well as pharmaceutically acceptable salts and solvates thereof.

The compounds of the present invention may be synthesized according to any technology or procedure known in the art, e.g., as described in detail with respect to compound 1 and depicted with respect to other compounds in the various schemes shown in the Examples section hereinafter.

Optically active forms of the compounds of the invention may be prepared using any method known in the art, e.g., by resolution of the racemic form by recrystallization techniques; by chiral synthesis; by extraction with chiral solvents; or by chromatographic separation using a chiral stationary phase. A non-limiting example of a method for obtaining optically active materials is transport across chiral membranes, i.e., a technique whereby a racemate is placed in contact with a thin membrane barrier, the concentration or pressure differential causes preferential transport across the membrane barrier, and separation occurs as a result of the non-racemic chiral nature of the membrane that allows only one enantiomer of the racemate to pass through. Chiral chromatography, including simulated moving bed chromatography, can also be used. A wide variety of chiral stationary phases are commercially available.

As shown in Example 5 hereinafter, using Universal calorimetric PARP-1 Assay Kit (Trevigen), compound 1 was found to be a potent PARP inhibitor with IC₅₀ of 26.20 nM. Furthermore, in a series of both in vitro and in vivo studies described in Examples 6-8, this compound was found to represent a new class of highly innovative cytoprotective agents, exhibiting a remarkable potency.

In an in vitro study described in Example 6, RAW cells were pre-treated for 4 hours with either compound 1 or ABT-888, a potent monofunctional PARP inhibitor (IC₅₀<5 nM), prior to exposure to H₂O₂ for 24 hours, and the potency of these two PARP inhibitors was compared. As found, while ABT-888 (0.1 μM) slightly restored viability (from 65% to 75%), compound 1 (0.1 μM) markedly increased viability (from 65% to 90%), indicating that although compound 1 is >4-fold weaker than ABT-888 as a PARP inhibitor, it more than doubled the extent of recovery of viability (25% vs. 10%) compared to ABT-888. These results, as well as the fact that in this same model system, lipoic acid had no beneficial effect at a concentration of 0.1 μM, clearly show that the conjugation of both PARP inhibitor and anti-oxidant moieties does not merely result in additional effects but confers new unexpected cytoprotective properties shared by neither alone.

Example 7 describes an in vivo study, in which the potency of compound 1 in treatment of an inflammation induced by zymosan was tested in CD mice. In particular, mice were first administered with zymosan (500 mg/kg IP), and then with compound 1 (60 mg/kg IP) at 1 hour after zymosan challenge, and 18 hours after zymosan administration, lung samples were scored for histologic injury and neutrophil concentration was determined by measurement of myeloperoxidase (MPO) activity. As found, administration of compound 1 an hour after zymosan challenge reduced elevations in lung MPO by 65% and diminished histologic injury by 80%, respectively, relative to vehicle control.

A further in vivo study is described in Example 8, in which the potency of compound 1 in treatment of chlorine inhalational lung injury was tested in Balb/c mice. In this particular study, mice were exposed in a cylindrical glass chamber to 400 ppm Cl₂ in air for 30 minutes, and 15 minutes after the conclusion of Cl₂ exposure, mice were initiated on a q12h regimen of compound 1 (30 mg/kg/dose IP in 0.5 ml D5W). At 24 hours, mice were euthanized, lung tissue was taken for examination of polymorphonuclear neutrophil (PMN) infiltration (as reflected by MPO) and lung histology, and as found, compound 1 therapy reduced the elevation in MPO and histological lung damage by 82% and 73%, respectively, relative to placebo (D5W).

In another aspect, the present invention thus relates to a pharmaceutical composition comprising a compound of the general formula A-X—B as defined above, or an enantiomer, diastereomer, racemate, or a pharmaceutically acceptable salt or solvate thereof, and a pharmaceutically acceptable carrier. In particular embodiments, the pharmaceutical composition of the invention comprises a compound selected from compounds 1-26, preferably compound 1, or an enantiomer, diastereomer, racemate, or a pharmaceutically acceptable salt or solvate thereof.

The compounds and pharmaceutical compositions of the present invention can be provided in a variety of formulations, e.g., in a pharmaceutically acceptable form and/or in a salt form, as well as in a variety of dosages.

In one embodiment, the pharmaceutical composition of the present invention comprises a non-toxic pharmaceutically acceptable salt of a compound of the general formula A-X—B. Suitable pharmaceutically acceptable salts include acid addition salts such as, without being limited to, the mesylate salt; the maleate salt, the fumarate salt, the tartrate salt, the hydrochloride salt, the hydrobromide salt, the esylate salt; the p-toluenesulfonate salt, the benzoate salt, the acetate salt, the phosphate salt, the sulfate salt, the citrate salt, the carbonate salt, and the succinate salt. Additional pharmaceutically acceptable salts include salts of ammonium (NH₄⁺) or an organic cation derived from an amine of the formula R₄N⁺, wherein each one of the R₅ independently is selected from H, C₁-C₂₂, preferably C₁-C₆ alkyl, such as methyl, ethyl, propyl, isopropyl, n-butyl, sec-butyl, isobutyl, tert-butyl, n-pentyl, 2,2-dimethylpropyl, n-hexyl, and the like, phenyl, or heteroaryl such as pyridyl, imidazolyl, pyrimidinyl, and the like, or two of the Rs together with the nitrogen atom to which they are attached form a 3-7 membered ring optionally containing a further heteroatom selected from N, S and O, such as pyrrolydine, piperidine and morpholine. Furthermore, where the compounds of the invention carry an acidic moiety, suitable pharmaceutically acceptable salts thereof may include metal salts such as alkali metal salts, e.g., lithium, sodium or potassium salts, and alkaline earth metal salts, e.g., calcium or magnesium salts.

Further pharmaceutically acceptable salts include salts of a cationic lipid or a mixture of cationic lipids. Cationic lipids are often mixed with neutral lipids prior to use as delivery agents. Neutral lipids include, but are not limited to, lecithins; phosphatidylethanolamine; diacyl phosphatidylethanolamines such as dioleoyl phosphatidylethanolamine, dipalmitoyl phosphatidylethanolamine, palmitoyloleoyl phosphatidylethanolamine and distearoyl phosphatidylethanolamine; phosphatidylcholine; diacyl phosphatidylcholines such as dioleoyl phosphatidylcholine, dipalmitoyl phosphatidylcholine, palmitoyloleoyl phosphatidylcholine and distearoyl phosphatidylcholine; phosphatidylglycerol; diacyl phosphatidylglycerols such as dioleoyl phosphatidylglycerol, dipalmitoyl phosphatidylglycerol and distearoyl phosphatidylglycerol; phosphatidylserine; diacyl phosphatidylserines such as dioleoyl- or dipalmitoyl phosphatidylserine; and diphosphatidylglycerols; fatty acid esters; glycerol esters; sphingolipids; cardiolipin; cerebrosides; ceramides; and mixtures thereof. Neutral lipids also include cholesterol and other 3β hydroxy-sterols.

Examples of cationic lipid compounds include, without being limited to, Lipofectin (Life Technologies, Burlington, Ontario) (1:1 (w/w) formulation of the cationic lipid N-[1-(2,3-dioleyloxy)propyl]-N,N,N-trimethylammonium chloride and dioleoylphosphatidyl-ethanolamine); Lipofectamine™ (Life Technologies, Burlington, Ontario) (3:1 (w/w) formulation of polycationic lipid 2,3-dioleyloxy-N-[2(spermine-carboxamido)ethyl]-N,N-dimethyl-1-propanamin-iumtrifluoroacetate and diol eoylphosphatidyl-ethanolamine), Lipofectamine Plus (Life Technologies, Burlington, Ontario) (Lipofectamine and Plus reagent), Lipofectamine 2000 (Life Technologies, Burlington, Ontario) (Cationic lipid), Effectene (Qiagen, Mississauga, Ontario) (Non liposomal lipid formulation), Metafectene (Biontex, Munich, Germany) (Polycationic lipid), Eu-fectins (Promega Biosciences, San Luis Obispo, Calif.) (ethanolic cationic lipids numbers 1 through 12: C₅₂H₁₀₆N₆O₄.4CF₃CO₂H, C₈₈H₁₇₈N₈O₄S₂.4CF₃CO₂H, C₄₀H₈₄NO₃P.CF₃CO₂H, C₅₀H₁₀₃N₇O₃.4CF₃CO₂H, C₅₅H₁₁₆N₈O₂.6CF₃CO₂H, C₄₉H₁₀₂N₆O₃.4CF₃CO₂H, C₄₄H₈₉N₅O₃.2CF₃CO₂H, C₁₀₀H₂₀₆N₁₂O₄S₂.8CF₃CO₂H, C₁₆₂H₃₃₀N₂₂O₉.13CF₃CO₂H, C₄₃H₈₈N₄O₂.2CF₃CO₂H, C₄₃H₈₈N₄O₃.2CF₃CO₂H, C₄₁H₇₈NO₈P); Cytofectene (Bio-Rad, Hercules, Calif.) (mixture of a cationic lipid and a neutral lipid), GenePORTER® (Gene Therapy Systems, San Diego, Calif.) (formulation of a neutral lipid (Dope) and a cationic lipid) and FuGENE 6 (Roche Molecular Biochemicals, Indianapolis, Ind.) (Multi-component lipid based non-liposomal reagent).

The pharmaceutically acceptable salts of the present invention may be formed by conventional means, e.g., by reacting a free base form of the active agent or ingredient, i.e., the compound of the present invention, with one or more equivalents of the appropriate acid in a solvent or medium in which the salt is insoluble, or in a solvent such as water which is removed in vacuo or by freeze drying, or by exchanging the anion/cation of an existing salt for another anion/cation on a suitable ion exchange resin.

The present invention encompasses solvates of the compounds of the invention as well as salts thereof, e.g., hydrates.

In one embodiment, the pharmaceutical composition of the present invention is formulated as nanoparticles.

The pharmaceutical compositions provided by the present invention may be prepared by conventional techniques, e.g., as described in Remington: The Science and Practice of Pharmacy, 19^{th} Ed., 1995. The compositions can be prepared, e.g., by uniformly and intimately bringing the active agent, i.e., the compound of the present invention, into association with a liquid carrier, a finely divided solid carrier, or both, and then, if necessary, shaping the product into the desired formulation. The compositions may be in solid, semisolid or liquid form and may further include pharmaceutically acceptable fillers, carriers, diluents or adjuvants, and other inert ingredients and excipients. The compositions can be formulated for any suitable route of administration, e.g., oral, nasogastric, nasoenteric, orogastric, parenteral (e.g., intramuscular, subcutaneous, intraperitoneal, intravenous, intraarterial or subcutaneous injection, or implant), gavage, buccal, nasal, sublingual or topical administration, as well as for inhalation. The dosage will depend on the state of the patient, and will be determined as deemed appropriate by the practitioner.

The pharmaceutical composition of the present invention may be in a form suitable for oral use, e.g., as tablets, troches, lozenges, aqueous, or oily suspensions, dispersible powders or granules, emulsions, hard or soft capsules, or syrups or elixirs. Compositions intended for oral use may be prepared according to any method known to the art for the manufacture of pharmaceutical compositions and may further comprise one or more agents selected from sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be, e.g., inert diluents such as calcium carbonate, sodium carbonate, lactose, calcium phosphate, or sodium phosphate; granulating and disintegrating agents, e.g., corn starch or alginic acid; binding agents, e.g., starch, gelatin or acacia; and lubricating agents, e.g., magnesium stearate, stearic acid, or talc. The tablets may be either uncoated or coated utilizing known techniques to delay disintegration and absorption in the gastrointestinal tract and thereby provide a sustained action over a longer period. For example, a time delay material such as glyceryl monostearate or glyceryl distearate may be employed. They may also be coated using the techniques described in the U.S. Pat. Nos. 4,256,108, 4,166,452 and 4,265,874 to form osmotic therapeutic tablets for control release. The pharmaceutical composition of the invention may also be in the form of oil-in-water emulsion.

The pharmaceutical composition of the present invention may be in the form of a sterile injectable aqueous or oleagenous suspension, which may be formulated according to the known art using suitable dispersing, wetting or suspending agents. The sterile injectable preparation may also be a sterile injectable solution or suspension in a non-toxic parenterally acceptable diluent or solvent. Acceptable vehicles and solvents that may be employed include, without limiting, water, Ringer's solution and isotonic sodium chloride solution.

The pharmaceutical compositions of the invention may be in any suitable form, e.g., tablets such as matrix tablets, in which the release of a soluble active agent is controlled by having the active diffuse through a gel formed after the swelling of a hydrophilic polymer brought into contact with dissolving liquid (in vitro) or gastro-intestinal fluid (in vivo). Many polymers have been described as capable of forming such gel, e.g., derivatives of cellulose, in particular the cellulose ethers such as hydroxypropyl cellulose, hydroxymethyl cellulose, methylcellulose or methyl hydroxypropyl cellulose, and among the different commercial grades of these ethers are those showing fairly high viscosity.

The pharmaceutical compositions of the present invention may comprise the active agent formulated for controlled release in microencapsulated dosage form, in which small droplets of the active agent are surrounded by a coating or a membrane to form particles in the range of a few micrometers to a few millimeters, or in controlled-release matrix.

Another contemplated formulation is depot systems, based on biodegradable polymers, wherein as the polymer degrades, the active agent is slowly released. The most common class of biodegradable polymers is the hydrolytically labile polyesters prepared from lactic acid, glycolic acid, or combinations of these two molecules. Polymers prepared from these individual monomers include poly (D,L-lactide) (PLA), poly (glycolide) (PGA), and the copolymer poly (D,L-lactide-co-glycolide) (PLG).

Pharmaceutical compositions according to the present invention, when formulated for inhalation, may be administered utilizing any suitable device known in the art, such as metered dose inhalers, liquid nebulizers, dry powder inhalers, sprayers, thermal vaporizers, electrohydrodynamic aerosolizers, and the like.

The compounds and pharmaceutical composition of the present invention are useful for prevention, treatment, or management of diseases, disorders and conditions associated with elevated PARP activity or expression. In fact, as known from the literature, animal models of various diseases, disorders or conditions associated with elevated PARP activity have shown that by inhibiting PARP activity or, alternatively, genetically deletion of one of the PARP isoforms, tissue injury or disfunction has been significantly reduced (Garcia Soriano et al., 2001; Jagtap et al., 2002; Komjáti et al., 2004; Liaudet et al. 2000; Mabley et al., 2001a; Mabley et al., 2001b; Murakami et al., 2004).

The term “treatment” as used herein with respect to a disease, disorder or condition associated with elevated PARP activity or expression refers to administration of a compound of the general formula A-X—B as defined above, or an enantiomer, diastereomer, racemate, or pharmaceutically acceptable salt or solvate thereof, after the onset of symptoms of said disease, disorder or condition. The term “prevention” as used herein with respect to said disease, disorder or condition refers to administration of said compound prior to the onset of symptoms, particularly to patients at risk for developing such symptoms; and the term “management” as used herein with respect to said disease, disorder or condition refers to prevention of recurrence of said disease, disorder or condition in a patient previously suffered from said disease, disorder or condition. The term “therapeutically effective amount” as used herein refers to the quantity of the compound of the general formula A-X—B as defined above, or an enantiomer, diastereomer, racemate, or pharmaceutically acceptable salt or solvate thereof, that is useful to treat, prevent or manage said disease, disorder or condition associated with elevated PARP activity or expression.

In one embodiment, the disease, disorder or condition associated with elevated PARP activity or expression is a disease, disorder or condition associated with ischemia-reperfusion injury. Non-limiting examples of such diseases, disorders or conditions include sepsis, septic shock, stroke, cataract formation, glaucoma, geographic atrophy, macular degeneration, angina, hemorrhagic shock, superantigen-induced circulatory shock, renal reperfusion injury, contrast agent-induced nephropathy, retinopathy of prematurity, necrotizing enterocolitis, neonatal respiratory distress syndrome, lung ischemia reperfusion injury, complications of IL-2 biotherapy, myocardial infarction, complications of cardiopulmonary bypass surgery, limb reperfusion injury, post-prostatectomy related erectile dysfunction, reperfusion complications related to vascular surgery including carotid endarterectomy, aortic aneurysm repair, peripheral arterial embolectomy and thrombectomy, crush injury, compartment syndrome, organ preservation, head trauma, and spinal cord injury

In another embodiment, the disease, disorder or condition associated with elevated PARP activity or expression is a neurodegenerative disease such as, without being limited to, Parkinson's disease, Alzheimer's disease, and amyotrophic lateral sclerosis.

In a further embodiment, the disease, disorder or condition associated with elevated PARP activity or expression is an inflammatory or immune disease. In certain particular embodiments, said inflammatory or immune disease is selected from sepsis, uveitis, rheumatoid arthritis, rheumatoid spondylitis, osteroarthritis, inflamed joints, eczema, inflammatory skin conditions, inflammatory eye conditions, conjunctivitis, tissue necrosis resulting from inflammation, tissue rejection following transplant surgery, graft vs. host disease, Crohn's disease and ulcerative colitis, airway inflammation, asthma, bronchitis, systemic lupus erythematosis, multiple sclerosis, glaucoma, smoking-induced lung injury, pulmonary fibrosis, pancreatitis, cardiomyopathy including chemotherapy-induced cardiomyopathy, complications of IL-2 biotherapy, diabetes, diabetic complications including diabetic retinopathy, peripheral neuropathy, acute macular degeneration, skin ulcers, renal disease, neumonia, mucositis, adult respiratory distress syndrome, smoke inhalation, and cutaneous burn injury. In other particular embodiments, said inflammatory or immune disease is an inflammatory disease of the lung caused by inhalation of toxic agents or irritants such as chlorine, phosgene, and smoke.

In still another embodiment, the disease, disorder or condition associated with elevated PARP activity or expression is cancer.

In still a further embodiment, the disease, disorder or condition associated with elevated PARP activity or expression is associated with radiation treatment of cancer.

In a further aspect, the present invention thus relates to a compound of the general formula A-X—B as defined above, preferably a compound selected from compounds 1-26, more preferably compound 1, or an enantiomer, diastereomer, racemate, or a pharmaceutically acceptable salt or solvate thereof, for use in the prevention, treatment, or management of a disease, disorder or condition associated with elevated PARP activity or expression.

In still a further aspect, the present invention provides a method for prevention, treatment, or management of a disease, disorder or condition associated with elevated PARP activity or expression, said method comprising administering to an individual in need a therapeutically effective amount of a compound of the general formula A-X—B as defined above, preferably a compound selected from compounds 1-26, more preferably compound 1, or an enantiomer, diastereomer, racemate, or a pharmaceutically acceptable salt or solvate thereof.

The invention will now be illustrated by the following non-limiting Examples.

### EXAMPLES

### Example 1

### Synthesis of 2-(4-(1,2-dithiolan-3-yl)butyl)-1H-benzo[d]imidazole-4-carboxamide, 1 (R-503)

2,3-diaminobenzamide bis-hydrochloride was prepared from 3-nitrophthalic acid according to a procedure previous described (Jufang et al., 2007), as depicted in Scheme 1 (steps a-e). In particular, dehydration of 3-nitrophthalic acid with neat acetic anhydride (step a) yielded the desired 3-nitrophthalic anhydride. The later experiment was run by slow addition of 3-nitrophthalic anhydride to an excess of ammonium hydroxide (step b), which yielded a mixture of ammonium salts of 2-carbamoyl-3-nitrobenzoic acid. The potassium salt of 2-carbamoyl-3-nitrobenzoic acid was then prepared and used in the subsequent Hofmann rearrangement, which was accomplished by addition of an amide to a solution of freshly prepared potassium hypobromite followed by heating (step c) yielding 2-amino-3-nitrobenzoic acid. The carboxylic acid in 2-amino-3-nitrobenzoic acid was converted to the corresponding acid chloride with thionyl chloride followed by an ammonium hydroxide quench (step d) to form 2-amino-3-nitrobenzamide. The nitro group was reduced with Raney nickel in ethyl acetate/ethanol (step e) to provide 2,3-diaminobenzamide, which was converted to the bis-hydrochloride salt for further reaction using HCl.

As depicted in Scheme 1 (steps f-g), a mixture of lipoic acid (1.030 gm) and 1,1′-carbonyldiimidazole (CDI; 1.2 gm) in DMF (6 ml) and pyridine (6 ml) was stirred at 45° C. for 30 minutes. 2,3-Diaminobenzamide bis-hydrochloride (1.3 gm) was then added and the mixture was stirred at room temperature for overnight (step D. The reaction mixture was concentrated and diluted with saturated sodium bicarbonate and ethyl acetate. Organic layer was separated, washed with water and brine, and dried on sodium sulfate. It was concentrated and the residue was passed over silica gel column and eluted with ethyl acetate. The solvent was removed under vacuum and the residue was dissolved in acetic acid (20 ml) (step g). The mixture was refluxed for 2 hours, and was then concentrated and diluted with saturated sodium bicarbonate and ethyl acetate. Organic layer was separated, washed with water and brine, and dried on sodium sulfate. The dried organic layer was concentrated, and the residue was passed through silica gel column and eluted with ethyl acetate. The desired fractions were collected and concentrated under vacuum to produce compound 1 (605 mg).

¹H-NMR: (DMSO-D⁶) 1.39-1.89 (m, 7H), 2.34-2.42 (m, 1H), 2.88 (t, J=7.6 Hz, 2H), 3.06-3.19 (m, 2H), 3.58-3.64 (m, 1H), 7.23 (t, J=7.6 Hz, 1H), 7.60-7.62 (dd, J=2.8 and 7.6 Hz, 1H), 7.65 (bs, 1H), 7.77-7.79 (dd, J=2.8 and 7.6 Hz, 1H), 9.31 (d, J=3.2 Hz, 1H), 12.66 (bs, 1H); MS (ES⁺): 322.5005 (M+1); Anal calc for C₁₅H₁₉N₃OS₂: C, 56.04%, H, 5.96%; N, 13.14%. Found: C, 56.32%, H, 6.12%, N, 13.14%.

The methanesulfonic acid (mesylate) salt of compound 1 was prepared from compound 1 (45 mg) and methane sulfonic acid (1.2 eq) in methanol (12 ml). The mixture was concentrated and washed with ether (3×5 ml); and the residue was dissolved in water (12 ml) and lyophilized to give 2-[4-(1,2-dithiolan-3-yl)butyl)-1H-benzo[d]imidazole-4-carboxamide methane sulfonic acid salt.

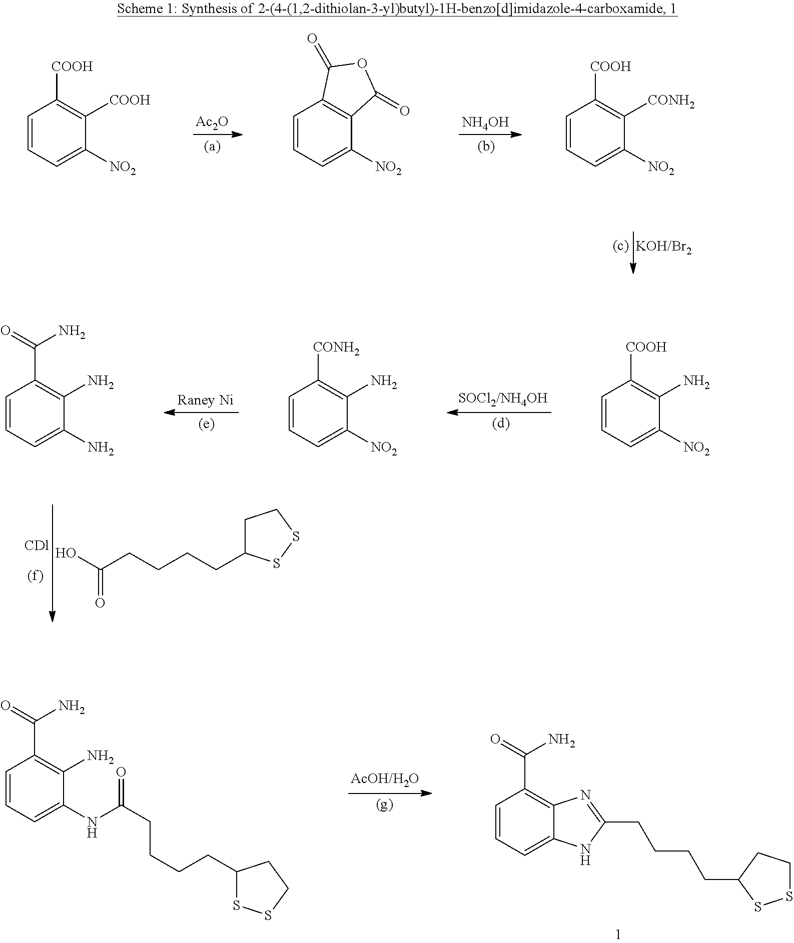

### Example 2

### Procedure for the synthesis of 2-(5-(1,2-dithiolan-3-yl)pentyl)-2H-indazole-7-carboxamide, 2

Compound 2 can be produced from 2-nitro-3-carboxymethylbenzaldehyde and 5-(1,2-dithiolan-3-yl)pentan-1-amine, as depicted in Scheme 2 below.

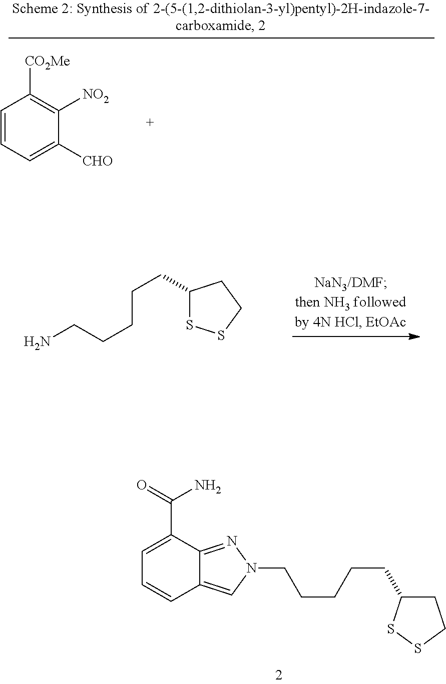

### Example 3

### Procedure for the synthesis of 2-(2-(5-(1,2-dithiolan-3-yl)pentyl)pyrrolidin-2-yl)-1H-benzo[d]imidazole-4-carboxamide, 3

Compound 3 can be produced as depicted in Scheme 3 below.

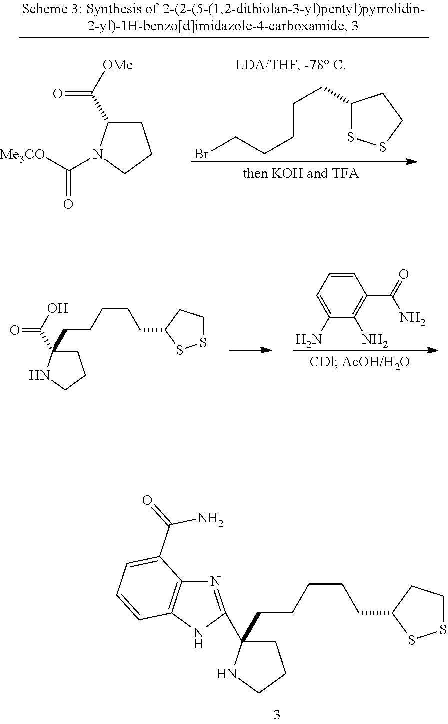

### Example 4

### Procedure for the synthesis of 3-(2-(4-carbamoyl-1H-benzo[d]imidazol-2-yl)pyrrolidin-2-yl)methyl)-2,2,5,5-tetramethylpyrrolidin-1-olate, 9

Compound 9 can be produced as depicted in Scheme 4 below.

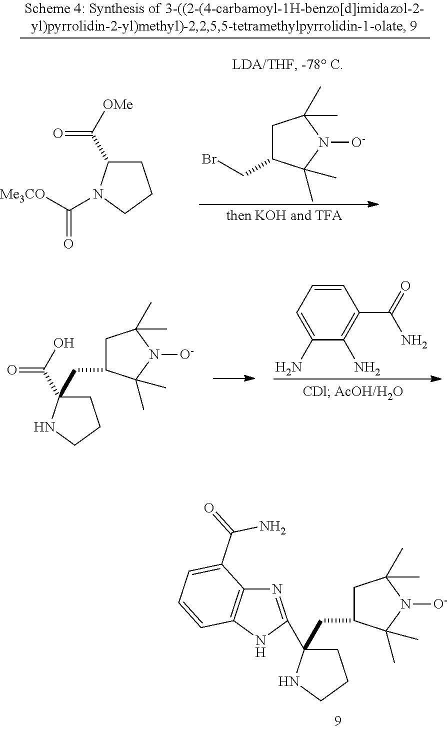

### Example 5

### Compound 1 is a Potent PARP Inhibitor

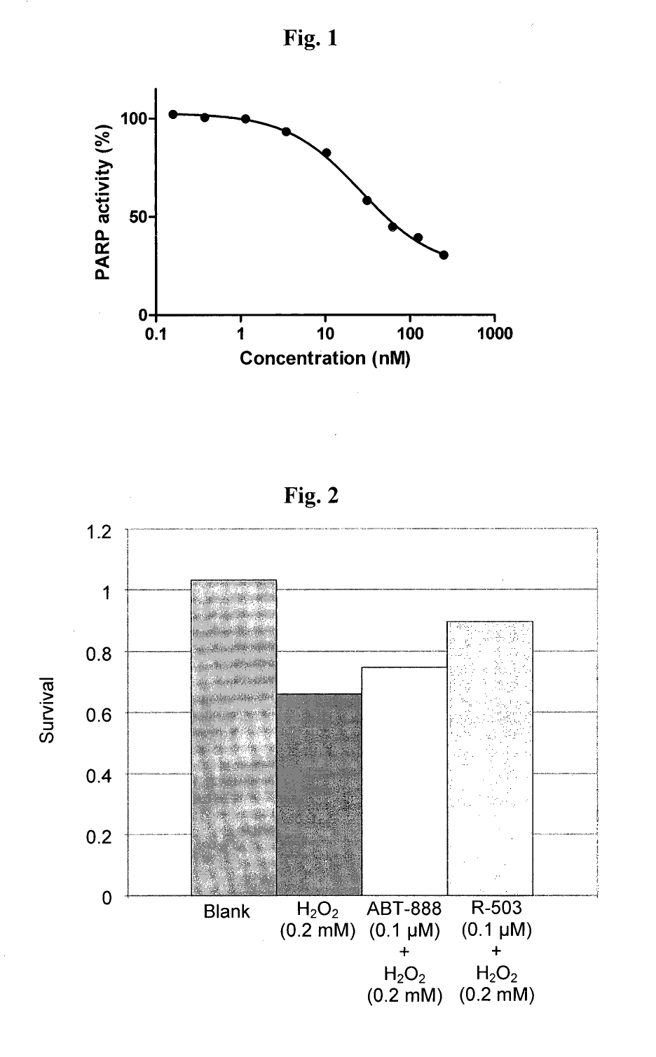

In this experiment, the PARP inhibition activity of compound 1 (R-503) was evaluated in in vitro potency assay using Universal calorimetric PARP-1 Assay Kit (Trevigen, Gaithersburg, Md.). Stock solutions (10 mM) of compound 1 were prepared in DMSO and diluted in water to a concentration range from 0.16 nM to 250 nM (10 point curve), and IC₅₀ values were calculated using Prizm software. As shown in FIG. 1, compound 1 is a potent PARP inhibitor with IC₅₀ of 26 nM.

### Example 6

### Compound 1 is a Superior Cytoprotective Agent than a Potent PARP Inhibitor

In this study, we have compared the potency of compound 1 (R-503) and ABT-888, a potent monofunctional PARP inhibitor (IC₅₀<5 nM), in a study in which RAW cells (a transformed murine macrophage cell line) were pre-treated for 4 hours with either compound 1 or ABT-888 prior to exposure to H₂O₂ (200 μM) for 24 hours. As shown in FIG. 2, while ABT-888 (0.1 μM) slightly restored viability (from 65% to 75%), compound 1 (0.1 μM) markedly increased viability (from 65% to 90%). Thus, despite the fact that compound 1 is >4-fold weaker than ABT-888 as a PARP inhibitor, it more than doubled the extent of recovery of viability (25% vs. 10%) compared to ABT-888. In this same model system, lipoic acid (LA) had no beneficial effect at a concentration of 0.1 μM (not shown). These results clearly show that the conjugation of both PARP inhibitor and anti-oxidant moieties does not merely result in additional effects but confers new unexpected cytoprotective properties shared by neither alone.

### Example 7

### Compound 1 is Effective in a Rodent Model of Inflammation

In this study, we tested the potency of compound 1 (R-503) in treatment of an inflammation induced by zymosan, a glucan with repeating glucose units connected by β-1,3-glycosidic linkages.

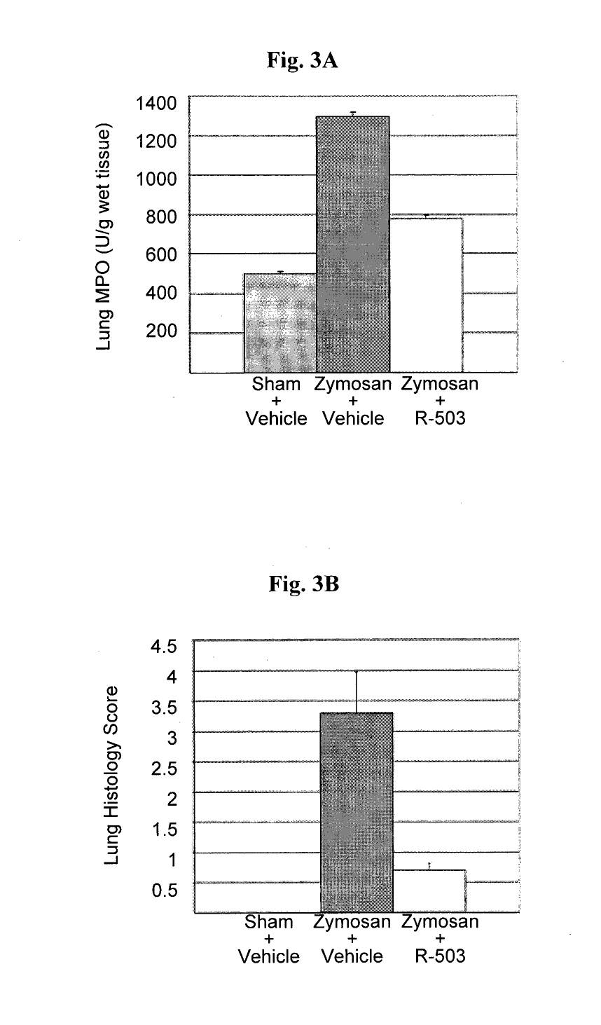

Male CD mice (20-22 g) were randomly allocated into the following groups (n=10 per experimental arm): (i) zymosan+vehicle group, wherein zymosan (500 mg/kg) was administered via an intraperitoneal (IP) route; (ii) zymosan+compound 1 group, which was identical to the zymosan+vehicle group except for the administration of compound 1 (60 mg/kg IP) at 1 hour after zymosan challenge; and (iii) Sham+vehicle group, which was identical to the zymosan+vehicle group but vehicle was administered in place of zymosan. 18 hours after zymosan admininstration, lung samples were scored for histologic injury by a pathologist blinded to group assignment and neutrophil concentration was determined by measurement of myeloperoxidase (MPO) activity. The following morphological criteria were used for scoring: 0, normal lung; grade 1, minimal edema or infiltration of alveolar or bronchiolar walls; grade 3, moderate edema and inflammatory cell infiltration without obvious damage to lung architecture; grade 4, severe inflammatory cell infiltration with obvious damage to lung architecture. As shown in FIGS. 3A-3B, addition of compound 1 an hour after zymosan challenge reduced elevations in lung MPO by 65% and diminished histologic injury by 80%, respectively.

### Example 8

### Compound 1 is Effective as a Rescue Therapy in a Murine Model of Chlorine Inhalational Lung Injury

In this study, we potency of compound 1 (R-503) in treatment of chlorine inhalational lung injury was tested.

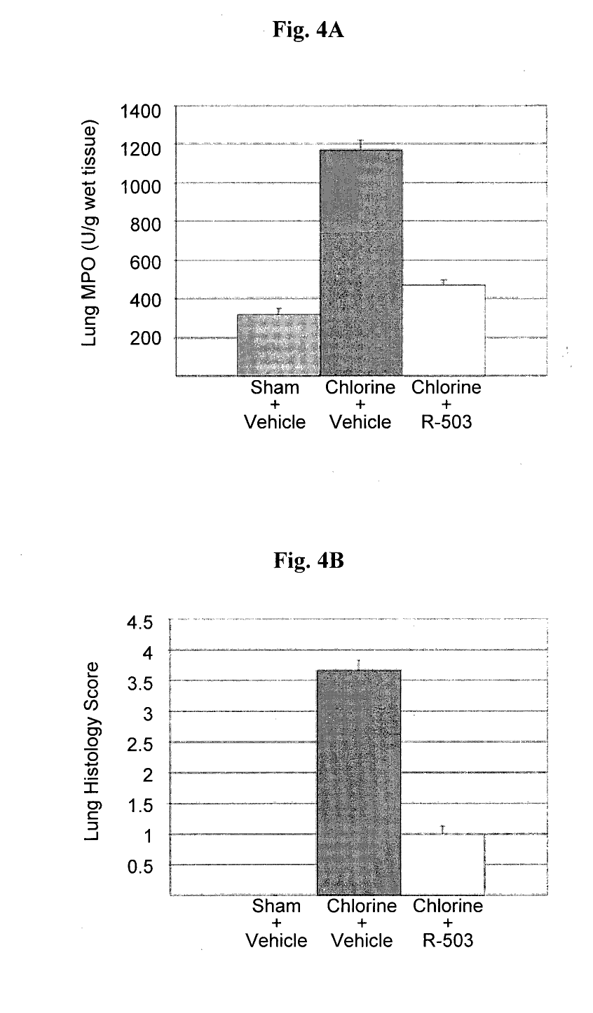

Male Balb/c mice were exposed in a cylindrical glass chamber to 400 ppm Cl₂ in air for 30 minutes. 15 minutes after the conclusion of Cl₂ exposure, mice were initiated on a q12h regimen of compound 1 (30 mg/kg/dose IP in 0.5 ml dextrose 5% in water, D5W). At 24 hours, mice were euthanized and lung tissue was taken for examination of polymorphonuclear neutrophil (PMN) infiltration (as reflected by MPO) and lung histology (H&E staining). The following morphological criteria were used for scoring: 0, normal lung; grade 1, minimal edema or infiltration of alveolar or bronchiolar walls; grade 3, moderate edema and inflammatory cell infiltration without obvious damage to lung architecture; grade 4, severe inflammatory cell infiltration with obvious damage to lung architecture. As shown in FIGS. 4A-4B, compound 1 therapy reduced the elevation in MPO and histological lung damage by 82% and 73%, respectively, relative to placebo (D5W).

### REFERENCES

Garcia Soriano F., Virág L., Jagtap P., Szabó E., Mabley J. G., Liaudet L., Marton A., Hoyt D. G., Murthy K. G., Salzman A. L., Southan G. J., Szabó C., Diabetic endothelial dysfunction: the role of poly(ADP-ribose) polymerase activation, Nat. Med., 2001, 7(1), 108-113
Ferraris D. V., Evolution of poly(ADP-ribose) polymerase-1 (PARP-1) inhibitors. From concept to clinic, J. Med. Chem., 2010, 53, 4561-4584
Jagtap P., Soriano F. G., Virág L., Liaudet L., Mabley J., Szabó E., Haskó G., Marton A., Lorigados C. B., Gallyas F. Jr., Sámegi B., Hoyt D. G., Baloglu E., VanDuzer J., Salzman A. L., Southan G. J., Szabó C., Novel phenanthridinone inhibitors of poly (adenosine 5′-diphosphate-ribose) synthetase: potent cytoprotective and antishock agents, Crit. Care Med., 2002, 30(5), 1071-1082
Jufang H., et al., Process Development for ABT-472, a Benzimidazole PARP Inhibitor, Org. Proc. Res. & Dev., 2007, 11, 693
Komjáti K., Mabley J. G., Virág L., Southan G. J., Salzman A. L., Szabó C., Poly(ADP-ribose) polymerase inhibition protect neurons and the white matter and regulates the translocation of apoptosis-inducing factor in stroke, Int J Mol. Med., 2004, 13(3), 373-382
Liaudet L., Soriano F. G., Szabó E., Virág L., Mabley J. G., Salzman A. L., Szabó C., Protection against hemorrhagic shock in mice genetically deficient in poly(ADP-ribose)polymerase, Proc Natl Acad Sci USA, 2000, 97(18), 10203-10208
Mabley J. G., Jagtap P., Perretti M., Getting S. J., Salzman A. L., Virág L., Szabó E., Soriano F. G., Liaudet L., Abdelkarim G. E., Haskó G., Marton A., Southan G. J., Szabó C., Anti-inflammatory effects of a novel, potent inhibitor of poly (ADP-ribose) polymerase, Inflamm Res., 2001a, 50(11), 561-569
Mabley J. G., Suarez-Pinzon W. L., Haskó G., Salzman A. L., Rabinovitch A., Kun E., Szabó C., Inhibition of poly (ADP-ribose) synthetase by gene disruption or inhibition with 5-iodo-6-amino-1,2-benzopyrone protects mice from multiple-low-dose-streptozotocin-induced diabetes, Br J Pharmacol., 2001b, 133(6), 909-919
Murakami K., Enkhbaatar P., Shimoda K., Cox R. A., Burke A. S., Hawkins H. K., Traber L. D., Schmalstieg F. C., Salzman A. L., Mabley J. G., Komjáti K., Pacher P., Zsengellér Z., Szabó C., Traber D. L., Inhibition of poly (ADP-ribose) polymerase attenuates acute lung injury in an ovine model of sepsis, Shock, 2004, 21(2), 126-133

1. A compound of the general formula:
A-X—B
or an enantiomer, diastereomer, racemate, or a pharmaceutically acceptable salt or solvate thereof,
wherein
A is a poly(ADP-ribose) polymerase (PARP) inhibitor moiety;
B is an anti-oxidant moiety selected from the group consisting of radicals (B₁)-(B₆): 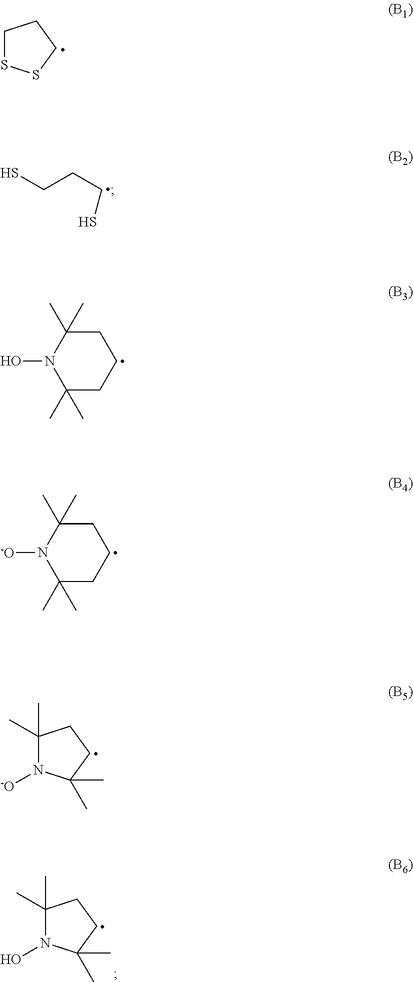
X is a covalent bond or represents one, two or three divalent moieties linked to each other, each independently selected from the group consisting of —O—, —S—, —CO—, —NH—, —NHCONH—, —(C₁-C₆)alkylene-, —N—(C₁-C₆)alkylene-, —(C₁-C₆)alkylene-O—CO—(C₁-C₆)alkylene-, —(C₁-C₆)alkylene-O—CO—, —(C₁-C₆)alkylene-NH—CO—(C₁-C₆)alkylene-, —(C₁-C₆)alkylene-NH—CO—, —O—(C₁-C₆)alkylene-, —O—CO—(C₁-C₆)alkylene-, —O—CO—, and a divalent cyclic radical selected from the group consisting of pyrrolidine-diyl, piperidine-diyl, (C₆-C₁₄)arylene-diyl, (C₄-C₁₂)cycloalkane-diyl, and 4-12-membered heterocyclic-diyl, wherein each one of said divalent cyclic radicals may be unsubstituted or substituted with one or more substituents each independently selected from the group consisting of halogen, —OH, —SH, —NH₂, —NO₂, (C₁-C₄)alkyl, —O—(C₁-C₄)alkyl and —S—(C₁-C₄)alkyl; and
the dot (•) represents the position of attachment to —X-A.

2. The compound of claim 1, wherein said PARP inhibitor moiety is a radical of the formula A₁, A₂ or A₃: 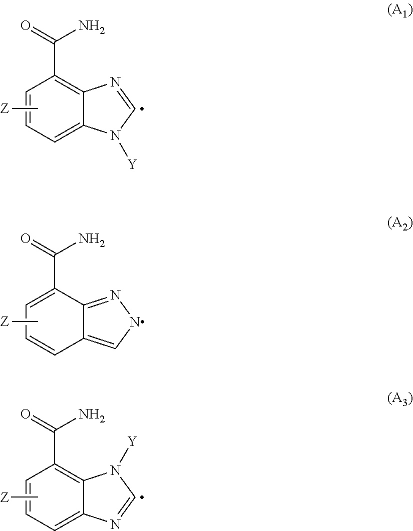
wherein
Y is selected from the group consisting of H, —OH, halogen, —CN, —(C₁-C₆)alkyl, —CO—(C₁-C₆)alkyl, —CO—O—(C₁-C₆)alkyl, —CO—(C₆-C₁₄)aryl, —CO-(4-12-membered heterocyclyl), —(C₃-C₈)monocyclic cycloalkyl, —N(R)₂, —(C₁-C₆)alkylene-N(R)₂, —N(Z)₂, —(C₁-C₆)alkylene-N(Z)₂, —S(O)₂—(C₁-C₆)alkyl, —S(O)₂NH—(C₁-C₆)alkyl, 3-8-membered heterocyclyl, and —(C₁-C₅)alkylene-(3-8-membered heterocyclyl), each of which other than —H, —OH, halogen and —CN is independently unsubstituted or substituted with one or more substituents each independently selected from the group consisting of halogen, —OH, —N(R)₂, —CF₃, —(C₁-C₆)alkyl, —O—(C₁-C₆)alkyl, —(C₆)aryl optionally substituted with at least one halogen, 3-7-membered heterocyclyl, —(C₁-C₆)alkylene-(C₆)aryl, —(C₁-C₆)alkylene-β-(C₁-C₆)alkyl, —C≡C—(C₁-C₄)alkyl-O—(C₁-C₆)alkyl, —(C₁-C₆)alkylene-OH, —(C₁-C₆)alkylene-N(R)₂, —(C₁-C₆)alkylene-CO—O—(C₁-C₆)alkyl, —CO—O—(C₁-C₆)alkyl, —CO—(C₁-C₆)alkylene-OH, —CO—N(R)₂, and —CO—(C₁-C₆)alkylene-N(R)₂;
R is independently H, (C₁-C₄)alkyl, (C₆)aryl, or 3-7-membered heterocyclyl;
Z is independently H, —OH —CN, —NO₂, halogen, —CH₃, —OCH₃, —CF₃ or —OCF₃; and
the dot (•) represents the position of attachment to —X—B.

3. The compound of claim 2, wherein said PARP inhibitor moiety is the radical of the formula A₁, wherein Y and Z are each H; the radical of the formula A₂, wherein Z is H; or the radical of the formula A₃, wherein Y and Z are each H.

4. The compound of claim 1, wherein said PARP inhibitor moiety is a moiety of a compound selected from the group consisting of compounds (A₄)-(A₁₄), which may be bound at any position to —X—B:

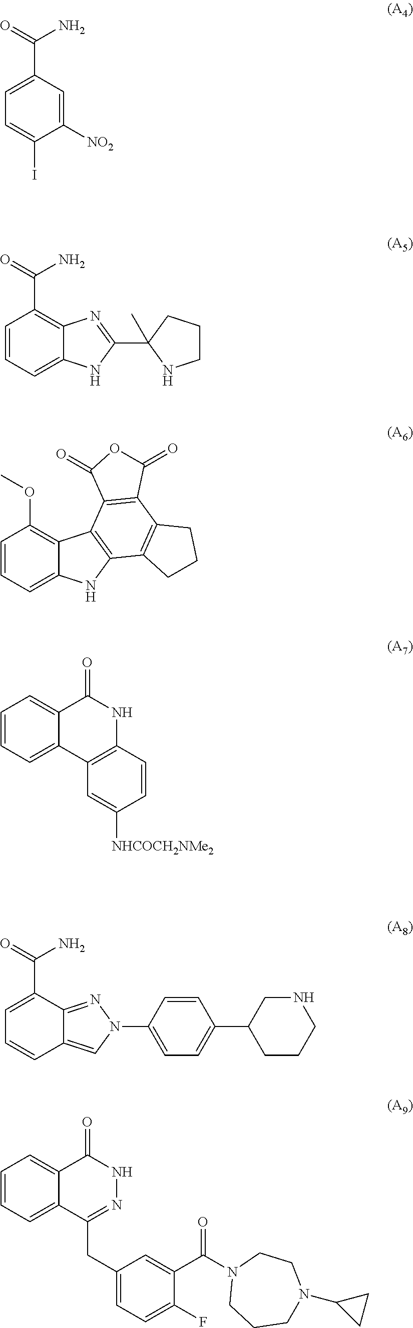

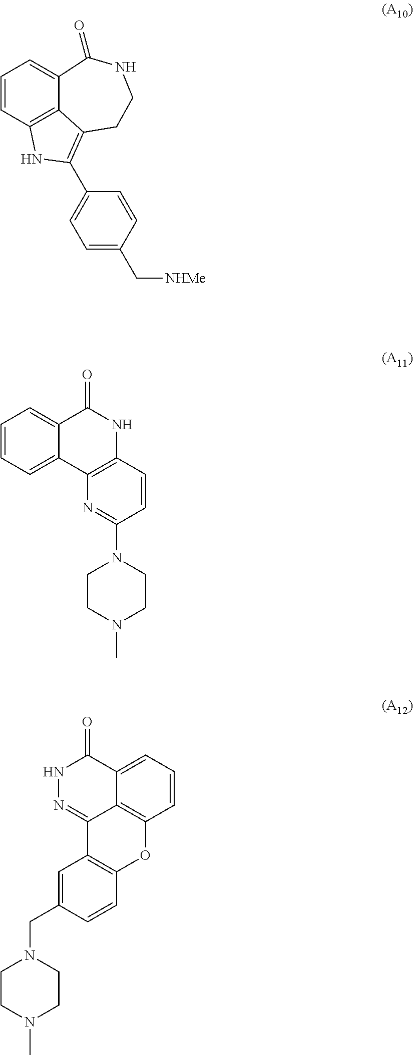 [SMILES: CNCc1ccc(-c2[nH]c3cccc4c3c2CCNC4=O)cc1 ; CN1CCN(c2ccc3[nH]c(=O)c4ccccc4c3n2)CC1 ; CN1CCN(Cc2ccc3c(c2)-c2n[nH]c(=O)c4cccc(c24)O3)CC1]

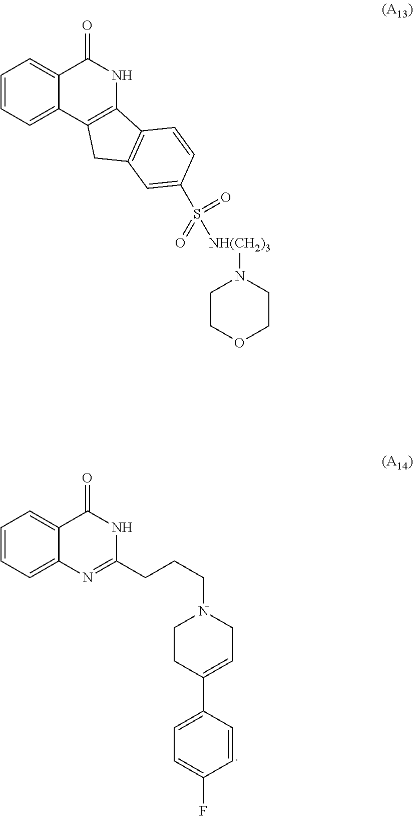

5. The compound of claim 1, wherein said PARP inhibitor is selected from the group consisting of benzamide derivatives, benzimidazole derivatives, phthalizinone derivatives, isoindolinone derivatives, phenanthridinone derivatives, and indenoisoquinolinone derivatives.

6. The compound of claim 1, wherein said PARP inhibitor moiety is a radical selected from the group consisting of radicals (A₁₅)-(A₂₁): 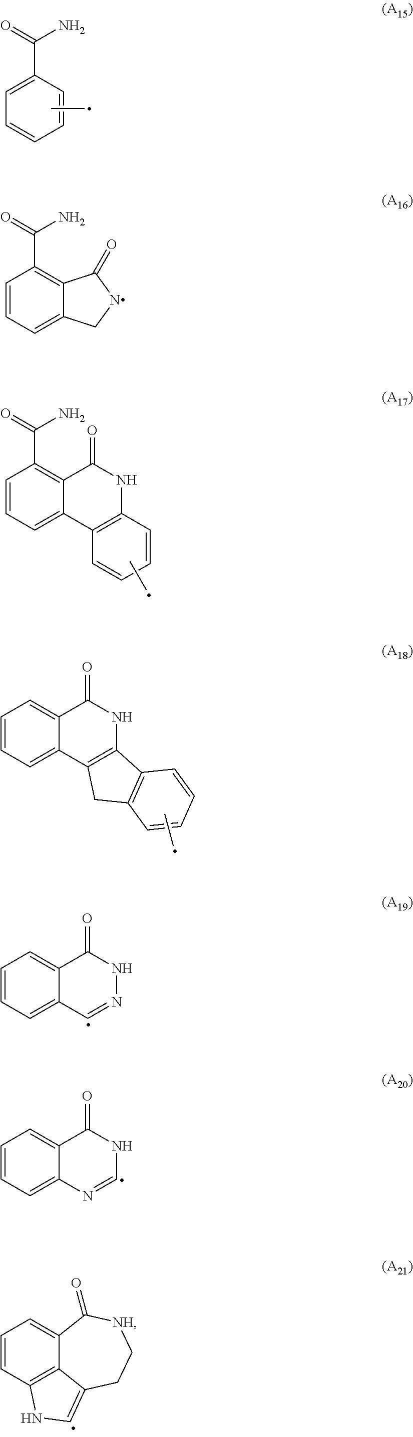
wherein the dot (•) represents the position of attachment to —X—B.

7. The compound of claim 1, wherein X represents:
one divalent moiety selected from the group consisting of —O—, —S—, CO—, —NH—, —NHCONH—, —(C₁-C₆)alkylene-, —N—(C₁-C₆)alkylene-, —(C₁-C₆)alkylene-O—CO—(C₁-C₆)alkylene-, —(C₁-C₆)alkylene-O—CO—, —(C₁-C₆)alkylene-NH—CO—(C₁-C₆)alkylene-, —(C₁-C₆)alkylene-NH—CO—, —O—(C₁-C₆)alkylene-, —O—CO—(C₁-C₆)alkylene-, and —O—C(O)—;
two divalent moieties linked to each other —Xₐ—X_{b}—, wherein Xₐ is selected from the group consisting of pyrrolidine-diyl, piperidine-diyl, (C₆-C₁₄)arylene-diyl, (C₄-C₁₂)cycloalkane-diyl, and 4-12-membered heterocyclic-diyl, optionally substituted with one or more substituents each independently selected from the group consisting of halogen, —OH, —SH, —NH₂, —NO₂, (C₁-C₄)alkyl, —O—(C₁-C₄)alkyl or —S—(C₁-C₄)alkyl; and X_{b} is —(C₁-C₆)alkylene-, —N—(C₁-C₆)alkylene-, —(C₁-C₆)alkylene-O—CO—(C₁-C₆)alkylene-, —(C₁-C₆)alkylene-O—CO—, —(C₁-C₆)alkylene-NH—CO—(C₁-C₆)alkylene-, —(C₁-C₆)alkylene-NH—CO—, —O—(C₁-C₆)alkylene-, —O—CO—(C₁-C₆)alkylene-, or —O—C(O)—; or
three divalent moieties linked to each other —Xₐ—X_{b}—X_{c}—, wherein Xₐ and X_{b} each independently selected from the group consisting of pyrrolidine-diyl, piperidine-diyl, (C₆-C₁₄)arylene-diyl, (C₄-C₁₂)cycloalkane-diyl or 4-12-membered heterocyclic-diyl, optionally substituted with one or more substituents each independently selected from the group consisting of halogen, —OH, —SH, —NH₂, —NO₂, (C₁-C₄)alkyl, —O—(C₁-C₄)alkyl or —S—(C₁-C₄)alkyl; and X_{c} is —(C₁-C₆)alkylene-, —N—(C₁-C₆)alkylene-, —(C₁-C₆)alkylene-O—CO—(C₁-C₆)alkylene-, —(C₁-C₆)alkylene-O—CO—, —(C₁-C₆)alkylene-NH—CO—(C₁-C₆)alkylene-, —(C₁-C₆)alkylene-NH—CO—, —O—(C₁-C₆)alkylene-, —O—CO—(C₁-C₆)alkylene-, or —O—C(O)—.

8. The compound of claim 7, wherein X represents:
a —(C₁-C₆)alkylene;
two divalent moieties linked to each other —Xₐ—X_{b}—, wherein Xₐ is a pyrrolidine-diyl; and X_{b} is —(C₁-C₆)alkylene-, —(C₁-C₆)alkylene-O—CO—(C₁-C₆)alkylene-, —O—(C₁-C₆)alkylene-, —O—CO—(C₁-C₆)alkylene-, or —O—C(O)—; or
three divalent moieties linked to each other —Xₐ—X_{b}—X_{c}—, wherein Xₐ is (C₆-C₁₄)arylene, preferably substituted with halogen; X_{b} is a piperidine-diyl; and X, is —(C₁-C₆)alkylene-O—CO—(C₁-C₆)alkylene-, —(C₁-C₆)alkylene-O—CO—, —(C₁-C₆)alkylene-NH—CO—(C₁-C₆)alkylene-, or —(C₁-C₆)alkylene-NH—CO—.

9. The compound of claim 8, wherein:
X is —(CH₂)₄— or —(CH₂)₅— (herein identified linkers X₁ and X₂, respectively);
X represents two divalent moieties linked to each other —Xₐ—X_{b}—, wherein Xₐ is 2,2-pyrrolidine-diyl; and X_{b} is —CH₂—, —(CH₂)₅— or —CH₂—O—CO—(CH₂)₄— (herein identified linkers X₃, X₄ and X₅, respectively);
X represents two divalent moieties linked to each other —Xₐ—X_{b}—, wherein Xₐ is 2,3-pyrrolidine-diyl; and X_{b} is selected from the group consisting of —O—CH₂—, —O—(CH₂)₅—, —O—CO— or —O—CO—(CH₂)₄—, linked at position 3 of the 2,3-pyrrolidine-diyl (herein identified linkers X₆, X₇, X₈ and X₉, respectively);
X represents two divalent moieties linked to each other —Xₐ—X_{b}—, wherein Xₐ is 2,4-pyrrolidine-diyl; and X_{b} is selected from the group consisting of —O—CH₂—, —O—(CH₂)₅—, —O—CO— or —O—CO—(CH₂)₄—, linked at position 4 of the 2,4-pyrrolidine-diyl (herein identified linkers X₁₀, X₁₁, X₁₂ and X₁₃, respectively); or
X represents three divalent moieties linked to each other —Xₐ—X_{b}—X_{c}—, wherein Xₐ is 3-fluoro-1,4 phenylene; X_{b} is 2,6-piperidine-diyl linked at position 1 of the 3-fluoro-1,4 phenylene; and X, is selected from the group consisting of —CH₂—O—CO—, —CH₂—O—CO—(CH₂)₄—, —CH₂—NH—CO— or —CH₂—NH—CO—(CH₂)₄—, linked at position 6 of the 2,6-piperidine-diyl (herein identified linkers X₁₄, X₁₅, X₁₆ and X₁₇, respectively).

10. The compound of claim 2, wherein (i) A is radical A₁ and B is radical B₁; (ii) A is radical A₁ and B is radical B₅; (iii) A is radical A₁ and B is radical B₄; (iv) A is radical A₂ and B is radical B₁; or (v) A is radical A₂ and B is radical B₅.

11. The compound of claim 10, wherein:
A is radical A₁ wherein both Y and Z are H; B is radical B₁; and X is —(CH₂)₄— (herein identified compound 1);
A is radical A₂ wherein Z is H; B is radical B₁; and X is —(CH₂)₅— (herein identified compound 2);
A is radical A₁ wherein both Y and Z are H; B is radical B₁; and X represents two divalent moieties linked to each other —Xₐ—X_{b}—, wherein Xₐ is 2,2-pyrrolidine-diyl; and X_{b} is —(CH₂)₅— (herein identified compound 3);
A is radical A₁ wherein both Y and Z are H; B is radical B₁; and X represents two divalent moieties linked to each other —Xₐ—X_{b}—, wherein Xₐ is 2,2-pyrrolidine-diyl; and X_{b} is —CH₂—O—CO—(CH₂)₄— (herein identified compound 4);
A is radical A₁ wherein both Y and Z are H; B is radical B₁; and X represents two divalent moieties linked to each other —Xₐ—X_{b}—, wherein Xₐ is 2,3-pyrrolidine-diyl; and X_{b} is —O—(CH₂)₅— linked at position 3 of the 2,3-pyrrolidine-diyl (herein identified compound 5);
A is radical A₁ wherein both Y and Z are H; B is radical B₁; and X represents two divalent moieties linked to each other —Xₐ—X_{b}—, wherein Xₐ is 2,3-pyrrolidine-diyl; and X_{b} is —O—CO—(CH₂)₄— linked at position 3 of the 2,3-pyrrolidine-diyl (herein identified compound 6);
A is radical A₁ wherein both Y and Z are H; B is radical B₁; and X represents two divalent moieties linked to each other —Xₐ—X_{b}—, wherein Xₐ is 2,4-pyrrolidine-diyl; and X_{b} is —O—(CH₂)₅— linked at position 4 of the 2,4-pyrrolidine-diyl (herein identified compound 7);
A is radical A₁ wherein both Y and Z are H; B is radical B₁; and X represents two divalent moieties linked to each other —Xₐ—X_{b}—, wherein Xₐ is 2,4-pyrrolidine-diyl; and X_{b} is —O—CO—(CH₂)₄— linked at position 4 of the 2,4-pyrrolidine-diyl (herein identified compound 8);
A is radical A₁ wherein both Y and Z are H; B is radical B₅; and X represents two divalent moieties linked to each other —Xₐ—X_{b}—, wherein Xₐ is 2,2-pyrrolidine-diyl; and X_{b} is —CH₂— (herein identified compound 9);
A is radical A₁ wherein both Y and Z are H; B is radical B₅; and X represents two divalent moieties linked to each other —Xₐ—X_{b}—, wherein Xₐ is 2,3-pyrrolidine-diyl; and X_{b} is —O—CH₂— linked at position 3 of the 2,3-pyrrolidine-diyl (herein identified compound 10);
A is radical A₁ wherein both Y and Z are H; B is radical B₅; and X represents two divalent moieties linked to each other —Xₐ—X_{b}—, wherein Xₐ is 2,3-pyrrolidine-diyl; and X_{b} is —O—CO— linked at position 3 of the 2,3-pyrrolidine-diyl (herein identified compound 11);
A is radical A₁ wherein both Y and Z are H; B is radical B₅; and X represents two divalent moieties linked to each other —Xₐ—X_{b}—, wherein Xₐ is 2,4-pyrrolidine-diyl; and X_{b} is —O—CH₂— linked at position 4 of the 2,4-pyrrolidine-diyl (herein identified compound 12);
A is radical A₁ wherein both Y and Z are H; B is radical B₅; and X represents two divalent moieties linked to each other —Xₐ—X_{b}—, wherein Xₐ is 2,4-pyrrolidine-diyl; and X_{b} is —O—CO— linked at position 4 of the 2,4-pyrrolidine-diyl (herein identified compound 13);
A is radical A₁ wherein both Y and Z are H; B is radical B₄; and X represents two divalent moieties linked to each other —Xₐ—X_{b}—, wherein Xₐ is 2,2-pyrrolidine-diyl; and X_{b} is —CH₂— (herein identified compound 14);
A is radical A₁ wherein both Y and Z are H; B is radical B₄; and X represents two divalent moieties linked to each other —Xₐ—X_{b}—, wherein Xₐ is 2,3-pyrrolidine-diyl; and X_{b} is —O—CH₂— linked at position 3 of the 2,3-pyrrolidine-diyl (herein identified compound 15);
A is radical A₁ wherein both Y and Z are H; B is radical B₄; and X represents two divalent moieties linked to each other —Xₐ—X_{b}—, wherein Xₐ is 2,3-pyrrolidine-diyl; and X_{b} is —O—CO— linked at position 3 of the 2,3-pyrrolidine-diyl (herein identified compound 16);
A is radical A₁ wherein both Y and Z are H; B is radical B₄; and X represents two divalent moieties linked to each other —Xₐ—X_{b}—, wherein Xₐ is 2,4-pyrrolidine-diyl; and X_{b} is —O—CH₂— linked at position 4 of the 2,4-pyrrolidine-diyl (herein identified compound 17);
A is radical A₁ wherein both Y and Z are H; B is radical B₄; and X represents two divalent moieties linked to each other —Xₐ—X_{b}—, wherein Xₐ is 2,4-pyrrolidine-diyl; and X_{b} is —O—CO— linked at position 4 of the 2,4-pyrrolidine-diyl (herein identified compound 18);
A is radical A₁ wherein both Y and Z are H; B is radical B₁; and X represents three divalent moieties linked to each other —Xₐ—X_{b}—X_{c}—, wherein Xₐ is 3-fluoro-1,4 phenylene; X_{b} is 2,6-piperidine-diyl linked at position 1 of the 3-fluoro-1,4 phenylene; and X_{c} is —CH₂—O—CO—(CH₂)₄— linked at position 6 of the 2,6-piperidine-diyl (herein identified compound 19);
A is radical A₁ wherein both Y and Z are H; B is radical B₁; and X represents three divalent moieties linked to each other —Xₐ—X_{b}—X_{c}—, wherein Xₐ is 3-fluoro-1,4 phenylene; X_{b} is 2,6-piperidine-diyl linked at position 1 of the 3-fluoro-1,4 phenylene; and X_{c} is —CH₂—NH—CO—(CH₂)₄— linked at position 6 of the 2,6-piperidine-diyl (herein identified compound 20);
A is radical A₁ wherein both Y and Z are H; B is radical B₅; and X represents three divalent moieties linked to each other —Xₐ—X_{b}—X_{c}—, wherein Xₐ is 3-fluoro-1,4 phenylene; X_{b} is 2,6-piperidine-diyl linked at position 1 of the 3-fluoro-1,4 phenylene; and X_{c} is —CH₂—O—CO— linked at position 6 of the 2,6-piperidine-diyl (herein identified compound 21);
A is radical A₁ wherein both Y and Z are H; B is radical B₅; and X represents three divalent moieties linked to each other —Xₐ—X_{b}—X_{c}—, wherein Xₐ is 3-fluoro-1,4 phenylene; X_{b} is 2,6-piperidine-diyl linked at position 1 of the 3-fluoro-1,4 phenylene; and X_{c} is —CH₂—NH—CO— linked at position 6 of the 2,6-piperidine-diyl (herein identified compound 22);
A is radical A₂ wherein Z is H; B is radical B₁; and X represents three divalent moieties linked to each other —Xₐ—X_{b}—X_{c}—, wherein Xₐ is 3-fluoro-1,4 phenylene; X_{b} is 2,6-piperidine-diyl linked at position 1 of the 3-fluoro-1,4 phenylene; and X_{c} is —CH₂—O—CO—(CH₂)₄— linked at position 6 of the 2,6-piperidine-diyl (herein identified compound 23);
A is radical A₂ wherein Z is H; B is radical B₁; and X represents three divalent moieties linked to each other —Xₐ—X_{b}—X_{c}—, wherein Xₐ is 3-fluoro-1,4 phenylene; X_{b} is 2,6-piperidine-diyl linked at position 1 of the 3-fluoro-1,4 phenylene; and X_{c} is —CH₂—NH—CO—(CH₂)₄— linked at position 6 of the 2,6-piperidine-diyl (herein identified compound 24);
A is radical A₂ wherein Z is H; B is radical B₅; and X represents three divalent moieties linked to each other —Xₐ—X_{b}—X_{c}—, wherein Xₐ is 3-fluoro-1,4 phenylene; X_{b} is 2,6-piperidine-diyl linked at position 1 of the 3-fluoro-1,4 phenylene; and X_{c} is —CH₂—O—CO— linked at position 6 of the 2,6-piperidine-diyl (herein identified compound 25);
A is radical A₂ wherein Z is H; B is radical B₅; and X represents three divalent moieties linked to each other —Xₐ—X_{b}—X_{c}—, wherein Xₐ is 3-fluoro-1,4 phenylene; X_{b} is 2,6-piperidine-diyl linked at position 1 of the 3-fluoro-1,4 phenylene; and X_{c} is —CH₂—NH—CO— linked at position 6 of the 2,6-piperidine-diyl (herein identified compound 26).

12. A pharmaceutical composition comprising a compound of claim 1, or an enantiomer, diastereomer, racemate, or a pharmaceutically acceptable salt or solvate thereof, and a pharmaceutically acceptable carrier.

13. The pharmaceutical composition of claim 12, wherein said compound is selected from the group consisting of the herein identified compounds 1-26.

14. The pharmaceutical composition of claim 12, for intravenous, intraarterial, intramuscular, subcutaneous, transdermal, nasal, oral, parenteral, rectal, vaginal, topical or ophthalmic topical administration, or for administration by inhalation.

15. The pharmaceutical composition of claim 12, formulated as a solid implant.

16. The pharmaceutical composition of claim 12, wherein said carrier comprises a biodegradable polymer.

17. The pharmaceutical composition of claim 16, formulated for slow release of the compound.

18-28. (canceled)

29. A method for prevention, treatment, or management of a disease, disorder or condition associated with elevated PARP activity or expression, said method comprising administering to an individual in need a therapeutic effective amount of a compound according to claim 1, or an enantiomer, diastereomer, racemate, or a pharmaceutically acceptable salt or solvate thereof.

30. The method of claim 29, wherein said disease, disorder or condition associated with elevated PARP activity or expression is a disease, disorder or condition associated with ischemia-reperfusion injury.

31. The method of claim 30, wherein said disease, disorder or condition associated with ischemia-reperfusion injury is selected from the group consisting of sepsis, septic shock, stroke, cataract formation, glaucoma, geographic atrophy, macular degeneration, angina, hemorrhagic shock, superantigen-induced circulatory shock, renal reperfusion injury, contrast agent-induced nephropathy, retinopathy of prematurity, necrotizing enterocolitis, neonatal respiratory distress syndrome, lung ischemia reperfusion injury, complications of IL-2 biotherapy, myocardial infarction, complications of cardiopulmonary bypass surgery, limb reperfusion injury, post-prostatectomy related erectile dysfunction, reperfusion complications related to vascular surgery including carotid endarterectomy, aortic aneurysm repair, peripheral arterial embolectomy and thrombectomy, crush injury, compartment syndrome, organ preservation, head trauma, and spinal cord injury.

32. The method of claim 29, wherein said disease, disorder or condition associated with elevated PARP activity or expression is a neurodegenerative disease.

33. The method of claim 32, wherein said neurodegenerative disease is Parkinson's disease, Alzheimer's disease, or amyotrophic lateral sclerosis.

34. The method of claim 29, wherein said disease, disorder or condition associated with elevated PARP activity or expression is an inflammatory or immune disease.

35. The method of claim 34, wherein said inflammatory or immune disease is selected from the group consisting of sepsis, uveitis, rheumatoid arthritis, rheumatoid spondylitis, osteroarthritis, inflamed joints, eczema, inflammatory skin conditions, inflammatory eye conditions, conjunctivitis, tissue necrosis resulting from inflammation, tissue rejection following transplant surgery, graft vs. host disease, Crohn's disease and ulcerative colitis, airway inflammation, asthma, bronchitis, systemic lupus erythematosis, multiple sclerosis, glaucoma, smoking-induced lung injury, pulmonary fibrosis, pancreatitis, cardiomyopathy including chemotherapy-induced cardiomyopathy, complications of IL-2 biotherapy, diabetes, diabetic complications including diabetic retinopathy, peripheral neuropathy, acute macular degeneration, skin ulcers, renal disease, neumonia, mucositis, adult respiratory distress syndrome, smoke inhalation, and cutaneous burn injury; or said inflammatory or immune disease is an inflammatory disease of the lung caused by inhalation of toxic agents or irritants such as chlorine, phosgene and smoke inhalation injury.

36. The method of claim 29, wherein said disease, disorder or condition associated with elevated PARP activity or expression is cancer or is associated with radiation treatment of cancer.
